SEQUENCE LISTING (6 sequences)

SEQ ID NO: 1 - DNA, 951 bp, Mus musculus
atgcgccgggccagccgagactacggcaagtacctgcgcagctcggaggagatgggcagcggccccggcgtcccacacgagggtccgctgcaccccgcgccttctgcaccggctccggcgccgccacccgccgcctcccgctccatgttcctggccctcctggggctgggactgggccaggtggtctgcagcatcgctctgttcctgtactttcgagcgcagatggatcctaacagaatatcagaagacagcactcactgcttttatagaatcctgagactccatgaaaacgcaggtttgcaggactcgactctggagagtgaagacacactacctgactcctgcaggaggatgaaacaagcctttcagggggccgtgcagaaggaactgcaacacattgtggggccacagcgcttctcaggagctccagctatgatggaaggctcatggttggatgtggcccagcgaggcaagcctgaggcccagccatttgcacacctcaccatcaatgctgccagcatcccatcgggttcccataaagtcactctgtcctcttggtaccacgatcgaggctgggccaagatctctaacatgacgttaagcaacggaaaactaagggttaaccaagatggcttctattacctgtacgccaacatttgctttcggcatcatgaaacatcgggaagcgtacctacagactatcttcagctgatggtgtatgtcgttaaaaccagcatcaaaatcccaagttctcataacctgatgaaaggagggagcacgaaaaactggtcgggcaattctgaattccacttttattccataaatgttgggggatttttcaagctccgagctggtgaagaaattagcattcaggtgtccaacccttccctgctggatccggatcaagatgcgacgtactttggggctttcaaagttcaggacatagactga

SEQ ID NO: 2 - protein, 316 aa, Mus musculus
MRRASRDYGKYLRSSEEMGSGPGVPHEGPLHPAPSAPAPAPPPAASRSMFLALLGLGLGQVVCSIALFLYFRAQMDPNRISEDSTHCFYRILRLHENAGLQDSTLESEDTLPDSCRRMKQAFQGAVQKELQHIVGPQRFSGAPAMMEGSWLDVAQRGKPEAQPFAHLTINAASIPSGSHKVTLSSWYHDRGWAKISNMTLSNGKLRVNQDGFYYLYANICFRHHETSGSVPTDYLQLMVYVVKTSIKIPSSHNLMKGGSTKNWSGNSEFHFYSINVGGFFKLRAGEEISIQVSNPSLLDPDQDATYFGAFKVQDID

SEQ ID NO: 3 - DNA, 20 bp, Artificial Sequence
  Primer
ccgcacgacaaccgcaccat

SEQ ID NO: 4 - DNA, 20 bp, Artificial Sequence
  Primer
cgctccggcccacaaatctc

SEQ ID NO: 5 - DNA, 20 bp, Artificial Sequence
  Primer
tctccactcttctagttcct

SEQ ID NO: 6 - DNA, 19 bp, Artificial Sequence
  Primer
ttgggtcatttccacatgc

(19) United States
(12) Patent Application Publication
(10) Pub. No.: US 2003/0013651 A1
(43) Pub. Date: Jan. 16, 2003

(54) Stimulation of osteogenesis using rank ligand fusion proteins

(75) Inventors: Jonathan Lam, West Memphis, AR (US); F. Patrick Ross, Olivette, MO (US); Steven L. Teitelbaum, University City, MO (US)

Correspondence Address: SONNENSCHEIN NATH & ROSENTHAL, P.O. BOX 061080, WACKER DRIVE STATION, CHICAGO, IL, 60606-1080, US

(73) Assignee: Barnes-Jewish Hospital

(21) Appl. No.: 10/105,057

(22) Filed: Mar. 22, 2002

Related U.S. Application Data
(60) Provisional application No. 60/277,855, filed on Mar. 22, 2001.
(60) Provisional application No. 60/311,163, filed on Aug. 9, 2001.
(60) Provisional application No. 60/329,231, filed on Oct. 12, 2001.
(60) Provisional application No. 60/328,876, filed on Oct. 12, 2001.
(60) Provisional application No. 60/329,393, filed on Oct. 15, 2001.

(51) Int. Cl. A61K 38/17

(52) U.S. Cl. 514/12

(57) ABSTRACT

A method of enhancing bone formation comprising administering an effective amount of 1) an oligomeric complex of one or more of RANKL, a RANKL fusion protein or an analog, derivative or mimic thereof, 2) an osteogenic compound capable of enhancing activity of one or more intracellular proteins in osteoblasts or osteoblast precursors, wherein said activity is indicative of bone formation, or 3) an osteogenic compound capable of inactivating one or more phosphatases in osteoblasts or osteoblast precursors, wherein said inactivation is indicative of bone formation. The method also may be used to treat a disease or condition manifested at least in part by the loss of bone mass by administering to a patient a pharmaceutical composition comprising an oligomeric complex or osteogenic compound disclosed herein.

This application claims the benefit of U.S. Provisional Applications Ser. Nos. 60/277,855, 60/311,163, 60/329,231, 60/328,876, and 60/329,393, filed Mar. 22, 2001, Aug. 9, 2001, Oct. 12, 2001, Oct. 12, 2001, and Oct. 15, 2001, respectively, all of which are hereby incorporated herein by reference.

This invention was made in part with Government support under National Institutes of Health Grants AR32788, AR46123 and DE05413. The Government has certain rights in the invention.

### FIELD OF THE INVENTION

The present invention relates to methods for enhancing processes of bone formation by the administration of effective amounts of oligomeric complexes of one or more of RANKL, a RANKL fusion protein, analog, derivative, or mimic or osteogenic compounds capable of 1) enhancing activity of intracellular proteins in osteoblasts or osteoblast precursors, wherein said activity is indicative of bone formation, or 2) inactivating phosphatases in osteoblasts or osteoblast precursors, wherein said inactivation is indicative of bone formation.

The present invention further relates to treating, preventing or inhibiting bone loss or reduced bone formation caused by diseases such as osteoporosis. It further relates to enhancing fracture repair and promoting bone ingrowth into orthopedic implants or sites of bony fusion by facilitating bone formation via administration of oligomeric complexes or osteogenic compounds described herein.

The invention further provides compositions for stimulating bone formation.

### BACKGROUND OF THE INVENTION

Various conditions and diseases which manifest themselves in bone loss or thinning are a critical and growing health concern. It has been estimated that as many as 30 million Americans and 100 million worldwide are at risk for osteoporosis alone. Mundy et al., Science, 286: 1946-1949 (1999). Other conditions known to involve bone loss include juvenile osteoporosis, osteogenesis imperfecta, hypercalcemia, hyperparathyroidism, osteomalacia, osteohalisteresis, osteolytic bone disease, osteonecrosis, Paget's disease of bone, bone loss due to rheumatoid arthritis, inflammatory arthritis, osteomyelitis, corticosteroid treatment, metastatic bone diseases, periodontal bone loss, bone loss due to cancer, age-related loss of bone mass, and other forms of osteopenia. Additionally, new bone formation is needed in many situations, e.g., to facilitate bone repair or replacement for bone fractures, bone defects, plastic surgery, dental and other implantations and in other such contexts.

Bone is a dense, specialized form of connective tissue. Bone matrix is formed by osteoblast cells located at or near the surface of existing bone matrix. Bone is resorbed (eroded) by another cell type known as the osteoclast (a type of macrophage). These cells secrete acids, which dissolve bone minerals, and hydrolases, which digest its organic components. Thus, bone formation and remodeling is a dynamic process involving an ongoing interplay between the creation and erosion activities of osteoblasts and osteoclasts. Alberts, et al., Molecular Biology of the Cell, Garland Publishing, N.Y. (3rd ed. 1994), pp.1182-1186.

Present forms of bone loss therapy are primarily anti-resorptive, in that they inhibit bone resorption processes, rather than enhance bone formation. Among the agents which have been used or suggested for treatment of osteoporosis because of their claimed ability to inhibit bone resorption are estrogen, selective estrogen receptor modulators (SERM's), calcium, calcitriol, calcitonin (Sambrook, P. et al., N.Engl.J.Med. 328:1747-1753), alendronate (Saag, K. et al., N.Engl.J.Med. 339:292-299) and other bisphosphonates. Luckman et al., J. Bone Min. Res. 13, 581 (1998). However, anti-resorptives fail to correct the low bone formation rate frequently involved in net bone loss, and may have undesired effects relating to their impact on the inhibition of bone resorption/remodeling or other unwanted side effects.

A key development in the field of bone cell biology is the recent discovery that RANK ligand (RANKL, also known as osteoprotegerin ligand (OPGL), TNF-related activation induced cytokine (TRANCE), and osteoclast differentiation factor (ODF)), expressed on stromal cells, osteoblasts, activated T-lymphocytes and mammary epithelium, is the unique molecule essential for differentiation of macrophages into osteoclasts. Lacey, et al., Cell 93: 165-176 (1998)(Osteoprotegerin Ligand Is a Cytokine that Regulates Osteoclast Differentiation and Activation.) The cell surface receptor for RANKL is RANK, Receptor Activator of Nuclear Factor (NF)-kappa B. RANKL is a type-2 transmembrane protein with an intracellular domain of less than about 50 amino acids, a transmembrane domain of about 21 amino acids, and an extracellular domain of about 240 to 250 amino acids. RANKL exists naturally in transmembrane and soluble forms. The deduced amino acid sequence for at least the murine, rat and human forms of RANKL and variants thereof are known. See e.g., Anderson, et al., U.S. Pat. No. 6,017,729, Boyle, U.S. Pat. No. 5,843,678, and X u J. et al., J. Bone Min. Res. (2000/15:2178) which are incorporated herein by reference. RANKL (OPGL) has been identified as a potent inducer of bone resorption and as a positive regulator of osteoclast development. Lacey et al., supra.

In addition to its role as a factor in osteoclast differentiation and activation, RANKL has been reported to induce human dendritic cell (DC) cluster formation. Anderson et al., supra and mammary epithelium development J. Fata et al., “The osteoclast differentiation factor osteoprotegerin ligand is essential for mammary gland development,” Cell, 103:41-50 (2000). However, that RANKL could play a role in anabolic bone formation processes or could be used in methods to stimulate osteoblast proliferation or bone nodule mineralization was previously unknown and unexpected.

Accordingly, even though much has been discovered about osteoclasts and their manipulation for therapeutic purposes, not much is known about osteoblasts and bone formation. Thus, a need exists, in general, for methods for enhancing bone formation and preventing or inhibiting bone loss by stimulating anabolic processes, to a degree greater than coordinate resorption.

### SUMMARY OF THE INVENTION

Accordingly, among the objects of the present invention is the provision of methods and compositions which stimulate osteogenesis, including enhanced activity of osteoblasts, commitment of osteoblast precursors to the osteoblast phenotype and in vivo bone matrix deposition. Thus, methods are provided for enhancing bone formation as well as for treating diseases and conditions of bone loss by increasing bone formation, whether or not bone resorption processes are otherwise affected.

Briefly, therefore, the present invention is directed toward a method of enhancing bone formation. The method calls for administering effective amounts of 1) oligomeric complexes of one or more of RANKL, a RANKL fusion protein, analog, derivative, or mimic, 2) osteogenic compounds capable of enhancing activity of intracellular proteins in osteoblasts or osteoblast precursors, wherein said activity is indicative of bone formation, or 3) osteogenic compounds capable of inactivating phosphatases in osteoblasts or osteoblast precursors, wherein said inactivation is indicative of bone formation

Also provided is a method of treating a disease or condition manifested at least in part by the loss of bone mass. The method comprises administering a pharmaceutical composition comprising a RANKL fusion protein or an analog, derivative or mimic thereof in an amount effective to promote bone formation. In another embodiment, a pharmaceutical composition comprising an osteogenic compound capable of enhancing activity of intracellular proteins in osteoblasts or osteoblast precursors, wherein said activity is indicative of bone formation may be used. In a further embodiment, a pharmaceutical composition comprising an osteogenic compound capable of inactivating phosphatases in osteoblasts or osteoblast precursors, wherein said inactivation is indicative of bone formation may be employed. The loss of bone mass is thereby prevented, inhibited or counteracted.

In another aspect, applicants have provided a composition for stimulating bone formation. The composition includes an effective amount of a RANKL fusion protein, oligomeric complex, or an analog, derivative or mimic thereof in a pharmaceutically acceptable carrier or excipient. Further provided are compositions which include effective amounts of osteogenic compounds in pharmaceutically acceptable carriers or excipients, wherein said osteogenic compounds are capable of 1) enhancing activity of intracellular proteins in osteoblasts or osteoblast precursors, wherein said activity is indicative of bone formation, or 2) inactivating phosphatases in osteoblasts or osteoblast precursors, wherein said inactivation is indicative of bone formation.

In one embodiment, intracellular proteins are selected from IKB-α and IKB-β. In a preferred embodiment, the intracellular proteins exhibiting prolonged activity comprise intracellular kinases, and more preferably such kinases are ERK1/2, IKK, PI3 kinase, Akt, JNK, and p38. In a more preferred embodiment, the kinases are ERK1/2.

In another preferred embodiment, the activity of one or more intracellular proteins constitutes phosphorylation of said protein(s). Specifically, the phosphorylated proteins include ERK1/2, IKK, P13 kinase, Akt, JNK, and p38. More preferably, the phosphorylated kinases are ERK1/2.

In another aspect, the activity of one or more intracellular proteins can be detected for at least about 15-30 minutes following the incubation of the osteogenic compound with osteoblasts or osteoblast precursors. Preferably, the activity can be detected for 40 minutes, and more preferably it can be detected for at least 60 minutes following said incubation.

In another embodiment, osteogenic compounds capable of inactivating one or more phosphatases in osteoblasts or osteoblast precursors, wherein said inactivation is indicative of bone formation may be used in the methods and compositions of the present invention. Preferably, said phosphatase is selected from the group consisting of ERK1-, ERK2-, IKK-, P13 kinase-, Akt-, JNK-, and p38-specific phosphatases, and more preferably the phosphatese is specific for ERK1/2. In another preferred embodiment, inactivation comprises phosphorylation of a phosphatase.

The preferred oligomeric complexes used in the methods and compositions described herein include oligomeric complexes of GST-RANKL, AP-RANKL, leucine zipper-RANKL, and RANKL derivative comprising the “flap” domain of TALL-1.

Other objects and features will be in part apparent and in part pointed out hereinafter.

### BRIEF DESCRIPTION OF FIGURES

FIG. 1 is the structure and sequence of the RANKL murine cDNA and protein used to produce the GST-RANKL fusion proteins discussed in Examples 1 and 25 below.

FIG. 2 depicts a size-exclusion chromatograph of the GST-RANKL fusion protein under conditions replicating the physiological milieu. See Example 1.

FIG. 3 is a histological presentation of GST-RANKL stimulation of bone formation ex vivo in whole calvarial organ culture, as discussed in Example 2. Arrows mark parietal bone thickness.

FIG. 4 is a graphic depiction of the dose-dependent increase in calvarial thickness due to GST-RANKL stimulation of bone formation in vitro, as discussed in Example 2. White bars indicate 1 dose exposure, whereas black bars indicate 2 dose exposure to GST-RANKL.

FIG. 5(a) is a histological presentation of GST-RANKL stimulation of bone formation in vivo in mice, shown at low power magnification, as discussed in Example 3.

FIG. 5(b) is a histological presentation of GST-RANKL stimulation of bone formation in vivo in mice, shown at high power magnification, as discussed in Example 3.

FIG. 5(c) depicts a dual-energy X-ray absorptiometry (DEXA) analysis of tibial metaphyses comparing bone mineral density of animals administered GST-RANKL or control vehicle in vivo, as discussed in Example 3. Scale bar=1 mm.

FIG. 6 is a histological presentation of a mouse tibia at high magnification, demonstrating in vivo activation of osteoblasts in animals administered GST-RANKL as discussed in Example 4. Arrow in the left panel indicates activated osteoblasts, whereas the arrow in the right panel indicates flat bone lining cells.

FIG. 7 is a graphical depiction of the impact of controlled administration of GST-RANKL to animals, illustrating the number of osteoclasts and activated osteoblasts, as discussed in Example 5. White bars indicate osteoclast numbers, whereas black bars indicate numbers of activated osteoblasts.

FIG. 8 is a histological presentation of GST-RANKL stimulation of mineralized bone nodule formation in marrow cells cultured ex vivo, as discussed in Example 6. Red histochemical reaction product represents mineralizing colony forming units of osteoblasts.

FIG. 9 is a depiction of an in vivo double fluorochrome label incorporation into mineralizing bone, as discussed in Example 4. MAR represents mineral apposition, BFR indicates bone formation, and (ex) and (en) indicate exocranial and endocranial surfaces of calvaria, respectively.

FIG. 10 is an image of a Western blot depicting the rapid activation of the members of the MAPK pathway in murine osteoclast precursors following the treatment of cells with GST-RANKL. The activity was measured at the time of GST-RANKL/RANK interaction (0 minutes) and 5, 15, and 30 minutes following the interaction. From the top, the second, fourth, and sixth panels show the total levels of JNK, p38, and ERK respectively. The first, third, and fifth panels depict the phosphorylated (activated) forms of JNK, p38, and ERK respectively.

FIG. 11 is an image of a Western blot depicting the activity of Akt in murine osteoclast precursors following the treatment of cells with GST-RANKL. The activation was monitored at the time of GST-RANKL/RANK interaction, and 5 and 15 minutes following the interaction. The bottom panel depicts the levels of total Akt at specified time points, whereas the top panel depicts the phosphorylated forms of Akt.

FIG. 12 is an image of a Western blot depicting the prolonged activity of the kinases in MAPK pathway in murine osteoblasts following the GST-RANKL treatment of cells compared to the treatment with RANKL alone. The time points for which the phosphorylation was measured included 0 minutes (time of GST-RANKL or RANKL stimulation of cells), and 5, 10, 20, 30, and 60 minutes after GST-RANKL/RANK or RANKL/RANK binding occurred. The kinases whose activity was measured included ERK, JNK, p38, and Akt. pERK designates phosphorylated ERK, ERK designates the total amount of the same protein, pJNK designates phosphorylated JNK, JNK designates the total amount of JNK, pp38 designates phosphorylated p38, p38 designates the total amount of p38, pAkt designates phosphorylated Akt, and Akt designates the total amount of the same protein. The first panel from the top is p-IkBα, which designates phosphorylated IkBα, whereas IkBα designates the total amount of the same protein.

FIG. 13 is an image of a Western blot depicting the prolonged activity of ERK1/2 in murine osteoblast precursors following the treatment of cells with GST-RANKL. The time points at which ERK1/2 activity was measured include 0, 5, 10, 20, 30, and 60 minutes following GST-RANKL/RANK interaction. pERK designates phosphorylated ERK whereas ERK designates the total amount of the same protein.

FIG. 14 is a graphic presentation of alkaline phosphatase (AP) activity following GST-RANKL exposure.

FIG. 15 depicts GST-RANKL as oligomeric complexes, whereas cleaved RANKL (GST removed) does not exist in oligmeric forms. (a) shows that cleaved RANKL migrates as a single trimeric species (1 n), while GST-RANKL exists as a polydisperse mixture of non-covalently associated mono-trimeric (1 n) and oligomeric (2-100 n) units under dynamic equlibrium. (b) depicts possible oligomeric structures.

FIG. 16 consists of confocal microscopy images showing that cleaved RANKL/RANK complexes are rapidly internalized, whereas GST-RANKL/RANK complexes remain on the cell surface for at least one hour. On the merged images, colocalization of RANK (green fluorescence) and cell surface (red fluorescence) appears yellow.

FIG. 17 is an image of an agarose gel depicting the expression of Type I collagen in response to GST-RANKL treatment. “+” indicates the treatment of primary osteoblasts with GST-RANKL, whereas “−” indicates the lack of such treatment. Osteoblasts were exposed to GST-RANKL for 1, 2, 4, or 6-hour exposures at the beginning of each successive 48-hour treatment window. All culltures harvested between 8-48 hours were exposed to GST-RANKL for 6 hours. β-actin expression is used as a control for the experiment.

FIG. 18 is an image of an agarose gel depicting the expression of Cbfa1 in the marrow of mice treated with GST-RANKL or GST alone (marked as “control”). The bottom panel is the experiment control, depicting the expression of HPRT (hypoxanthine phosphoribosyl transferase).

FIG. 19 is a graphic representation of osteoblast proliferation as measured by BrdU (5-bromo-2′-deoxyuridine) incorporation in response to GST-RANKL treatment.

FIG. 20(a) is an image of a Western blot showing that osteoblasts transduced with dominant-negative ERK fail to phosphorylate an ERK substrate, known as RSK. DN-ERK represents dominant-negative ERK. LacZ represents β-galactosidase.

FIG. 20(b) is an image of an agarose gel showing that osteoblasts transduced with dominant-negative ERK fail to upregulate the expression of type I collagen in response to GST-RANKL.

### ABBREVIATIONS AND DEFINITIONS

To facilitate understanding of the invention, a number of terms are defined below:

“MAP kinase” or “MAPK” are used interchangeably herein, and are abbreviations for mitogen activated protein kinase.

“ERK1/2” refers to ERK1 and ERK2, which are abbreviations for extracellular signal-regulated kinase 1 and extracellular signal-regulated kinase 2, respectively.

JNK is an abbreviation for c-jun N-terminal kinase.

p38 is a kinase of 38 kDa, which is a member of the MAPK family of kinases.

Akt is Akt serine threonine kinase.

“IKB” is an abbreviation for IkappaB protein. Thus, IKB-α is IkappaB α and IKB-β is IkappaB β.

“IKK” is an abbreviation for IkappaB (IKB) kinase.

“RSK” is an abbreviation for p90 ribosomal S6 protein kinase.

“RANKL” or “RANK ligand” are used interchangeably herein to indicate a ligand for RANK (Receptor Activator of NFκB).

“AP” is an abbreviation for alkaline phosphatase.

“GST” is an abbreviation for glutathione-s-transferase.

“HPRT” is an abrreviation for hypoxanthine phosphoribosyl transferase.

“Cbfa1” is an abbreviation for core binding factor 1.

“LacZ” is an abbreviation for β-galactosidase.

“Osteogenic potential” or “osteogenic activity” are used interchangeably herein to refer to any compound that is able to enhance bone formation, as determined from bone formation assays.

“BrdU” is an abbreviation for 5-bromo-2′-deoxyuridine.

“TALL-1” is an abbreviation for a protein “TNF-and APOL-related leukocyte expressed ligand 1”.

By the term “an effective amount” is meant an amount of the substance in question which produces a statistically significant effect. For example, an “effective amount” for therapeutic uses is the amount of the composition comprising an active compound herein required to provide a clinically significant increase in healing rates in fracture repair; reversal or inhibition of bone loss in osteoporosis; prevention or delay of onset of osteoporosis; stimulation and/or augmentation of bone formation in fracture non-unions and distraction osteogenesis; increase and/or acceleration of bone growth into prosthetic devices; repair or prevention of dental defects; or treatment or inhibition of other bone loss conditions, diseases or defects, including but not limited to those discussed herein above. Such effective amounts will be determined using routine optimization techniques and are dependent on the particular condition to be treated, the condition of the patient, the route of administration, the formulation, and the judgment of the practitioner and other factors evident to those skilled in the art. The dosage required for the compounds of the invention (for example, in osteoporosis where an increase in bone formation is desired) is manifested as that which induces a statistically significant difference in bone mass between treatment and control groups. This difference in bone mass may be seen, for example, as at least 1-2%, or any clinically significant increase in bone mass in the treatment group. Other measurements of clinically significant increases in healing may include, for example, an assay for the N-terminal propeptide of Type I collagen, tests for breaking strength and tension, breaking strength and torsion, 4-point bending, increased connectivity in bone biopsies and other biomechanical tests well known to those skilled in the art. General guidance for treatment regimens is obtained from the experiments carried out in animal models of the disease of interest.

As used herein, “treatment” includes both prophylaxis and therapy. Thus, in treating a subject, the compounds of the invention may be administered to a subject already suffering from loss of bone mass or to prevent or inhibit the occurrence of such condition.

### DETAILED DESCRIPTION OF THE INVENTION

In accordance with the present invention, applicants have discovered that oligomeric complexes of RANKL fusion proteins, particularly oligomers of GST-RANKL, or variants, analogs, derivatives and mimics thereof, can be administered in an amount and manner such that they stimulate a net increase in the numbers of activated osteoblasts and enhance the anabolic processes of bone formation. Such discovery provides the basis for methods useful to facilitate bone replacement or repair, as well as for treating diseases or conditions involving loss of bone mass by stimulating anabolic processes of bone formation.

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

All publications, patents, patent applications, databases and other references cited in this application are herein incorporated by reference in their entirety as if each individual publication, patent, patent application, database or other reference were specifically and individually indicated to be incorporated by reference.

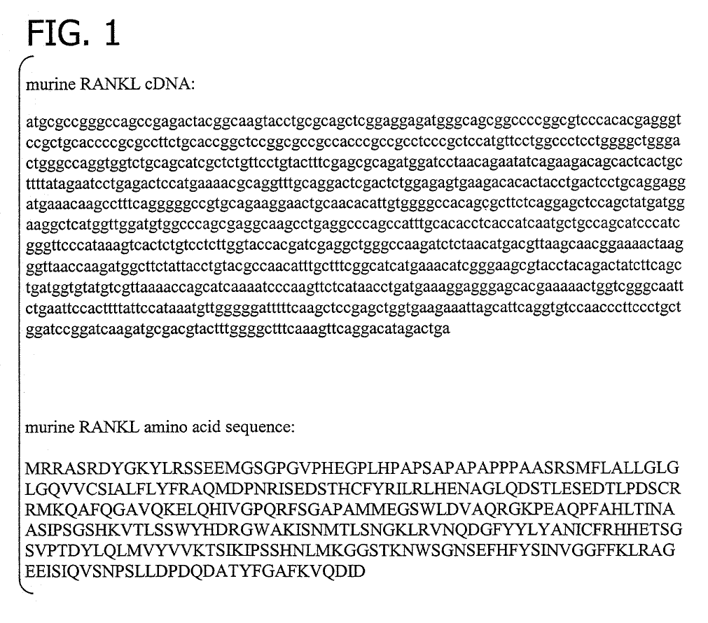

The selection and/or synthesis of RANKL, its fragments, variants, analogs, mimics, fusion products and oligomeric complexes of such compounds, wherein said oligomeric complexes are capable of promoting bone formation as taught herein, are within the ability of a person of ordinary skill in the art and are contemplated as being within the scope of this invention. For example, Boyle, supra, provides a detailed discussion of the synthesis of various forms of RANKL therein (called “osteoprotegerin binding protein”), and discloses, e.g., murine and human variants, recombinant forms of RANKL, RANKL fragments, analogs, mimics and derivatives of RANKL, and fusion-proteins thereof. Also included within the scope of the invention are derivatives or analogs of RANKL which have been modified post-translationally (such as glycosylated proteins), as well as polypeptides which are encoded by nucleic acids shown to hybridize to part or all of the polypeptide coding regions of RANKL cDNA under conditions of high stringency. See, e.g., Boyle and Anderson, et al., supra. The murine RANKL nucleic acid and amino acid sequences are provided herein as SEQ ID NO. 1 and SEQ ID NO. 2, respectively (see FIG. 1). However, RANKL sequences from other species have been identified and are available at http://www.ncbi.nlm.nih.gov/. Human RANKL nucleic acid and amino acid sequences have, for instance, the following accession numbers: AF019047 and AAB86811. Rat RANKL nucleic acid and amino acid sequences have, for example, these accession numbers: NM_{—}057149 and NP_{—}476490. Accordingly, any of the RANKL molecules may be used in the methods of the present invention, and are thus contemplated within the scope of the present invention.

RANKL and related molecules can be synthesized by using nucleic acid molecules which encode the peptides of this invention in an appropriate expression vector which include the encoding nucleotide sequences using procedures well known in the art. Such DNA molecules may be prepared, and subsequently analyzed, e.g., using automated DNA sequencing and the well-known codon-amino acid relationship of the genetic code. Such a DNA molecule also may be obtained as genomic DNA or as cDNA using oligonucleotide probes and conventional hybridization methodologies. Such DNA molecules may be incorporated into expression vectors, including plasmids, which are adapted for the expression of the DNA and production of the polypeptide in a suitable host such as bacterium, e.g., Escherichia coli, yeast cell, insect cell or mammalian cell. See, e.g., Examples 1 and 25. Methods for the production of such recombinant proteins, including fusion proteins, are well known in the art and can be found in standard molecular biology references such as Sambrook et al., Molecular Cloning, 2nd ed., Cold Spring Harbor Laboratory Press, 1989 and Ausubel et al., Current Protocols in Molecular Biology, 3rd ed., Wiley and Sons, 1995, and updates, incorporated herein by reference.

It is further known that certain modifications can be made without completely abolishing the polypeptide's activity. Modifications include the removal, substitution and addition of amino acids. Polypeptides containing other modifications can be synthesized by one skilled in the art. Thus, the effectiveness of the polypeptides can be modulated through various changes in the amino acid sequence or structure.

Further, it should be understood that the aforementioned analogs or mimics may be modified using methods known in the art to improve features such as solubility, safety, or efficacy. A necessary characteristic of these preferred compounds is the capability to stimulate bone formation when employed according to applicants' methods described herein.

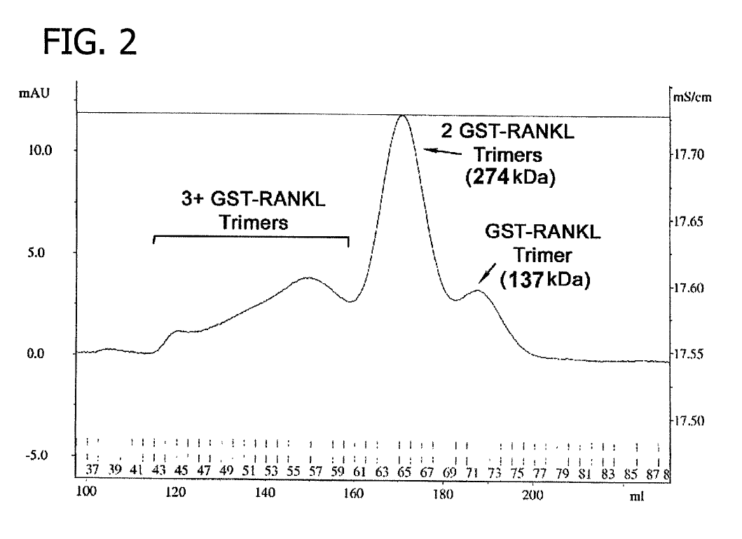

Applicants have discovered that administration of oligomers of GST-RANKL results in enhanced anabolic processes of bone formation. As shown in Example 1 and FIG. 2, size exclusion chromatography indicates that RANKL fusion proteins are capable of existing as oligomeric complexes under physiologic conditions. Oligomers of GST-RANKL are believed to be formed as a result of RANKL's and GST's tendencies to trimerize and dimerize, respectively. Accordingly, other fusion partners besides GST may be used to form oligomeric complexes comprising RANKL. Preferred fusion partners include alkaline phosphatase and leucine zippers, however any other proteins with a tendency to form oligomeric structures are contemplated within the scope of the present invention. In a preferred embodiment, RANKL fusion partners are added to the N-terminal of RANKL. Formation of GST-RANKL used to form oligomeric complexes is described in Examples 1 and 25. Furthermore, it is within the skill of the art to generate other forms of RANKL oligomers by well known techniques. For example, one could construct RANKL oligomers using alternative proteins or polypeptides that have an intrinsic tendency to self-associate and/or form higher-order complexes. One could also create such oligomers by chemical modification or by synthesizing a polymeric form of RANKL in which many copies are linked together, e.g., similar to a chain of pearls. Such alternative embodiments are also within the scope of this invention.

Alkaline phosphatase (AP), like GST, has a tendency to dimerize. APs form a large family of enzymes that are common to all organisms. Humans possess four isoforms of AP, three of which are tissue-specific and one which is non-specific and can be found in bone, liver, and kidney. The three tissue-specific APs include: placental AP (PLAP), germ cell AP (GCAP), and intestinal AP. The construction of an amino-terminal AP-RANKL may be performed similarly to the construction of GST-RANKL fusion protein. Examples of alkaline phosphatases that may be used include but are not limited to human placental AP-1, human placental AP-2, human placental AP precursor, mouse secreted AP, mouse embryonic AP precursor, and mouse embryonic AP with the corresponding accession numbers: AAA517110, AAA51707, AAC97139, AAL17657, P24823, and AAA37531. In one preferred embodiment, human placental alkaline phosphatase is employed, however other APs, isolated either from humans or from other mammalian species such as Mus musculus may be used. The use of many different alkaline phosphatases is believed to be feasible due to the ability of all APs to dimerize. Briefly, a cDNA encoding a desired isoform of AP can be isolated from a cDNA library and spliced upstream (at amino terminal) of a RANKL cDNA in a suitable expression vector, such as, e.g., pcDNA 3.1, using appropriate restriction endonucleases, such that the resulting DNA sequence is in frame, with no intervening stop codons. The expression vector, comprising the nucleotide sequence encoding AP-RANKL can then be introduced into host cells of choice by any of several trasfection or transduction techniques known in the art. See also Example 17.

Alternatively, a RANKL fusion protein may comprise a peptide with the ability to oligomerize, such as a leucine zipper domain. Leucine zippers were originally identified in several DNA-binding proteins (Landschulz et al., Science 240:1759, 1988). Leucine zipper domain is a term used to refer to a conserved peptide domain present in these (and other) proteins, which is responsible for dimerization of the proteins. The leucine zipper domain comprises a repetitive heptad repeat, with four or five leucine residues interspersed with other amino acids. Examples of leucine zipper domains are those found in the yeast transcription factor GCN4 and a heat-stable DNA-binding protein found in rat liver (C/EBP; Landschulz et al., Science 243:1681, 1989).

Leucine zipper domains are known to fold as short, parallel coiled coils. (O'Shea et al., Science 254:539; 1991) The general architecture of the parallel coiled coil has been well characterized, with a “knobs-into-holes” packing as proposed by Crick in 1953 (Acta Crystallogr. 6:689). The dimer formed by a leucine zipper domain is stabilized by the heptad repeat, designated (abcdefg)ₙ according to the notation of McLachlan and Stewart (J. Mol. Biol. 98:293; 1975), in which residues a and d are generally hydrophobic residues, with d being a leucine, which line up on the same face of a helix. Oppositely-charged residues commonly occur at positions g and e. Thus, in a parallel coiled coil formed from two helical leucine zipper domains, the “knobs” formed by the hydrophobic side chains of the first helix are packed into the “holes” formed between the side chains of the second helix.

Several studies have indicated that conservative amino acids may be substituted for individual leucine residues with minimal decrease in the ability to dimerize; multiple changes, however, usually result in loss of this ability (Landschulz et al., Science 243:1681,1989; Turner and Tjian, Science 243:1689,1989; Hu et al., Science 250:1400, 1990). van Heekeren et al. reported that a number of different amino residues can be substituted for the leucine residues in the leucine zipper domain of GCN4, and further found that some GCN4 proteins containing two leucine substitutions were weakly active (Nucl. Acids Res. 20:3721, 1992).

Amino acid substitutions in the a and d residues of a synthetic peptide representing the GCN4 leucine zipper domain have been found to change the oligomerization properties of the leucine zipper domain (Alber, Sixth Symposium of the Protein Society, San Diego, Calif.). When all residues at position a are changed to isoleucine, the leucine zipper still forms a parallel dimer. When, in addition to this change, all leucine residues at position d are also changed to isoleucine, the resultant peptide spontaneously forms a trimeric parallel coiled coil in solution. Substituting all amino acids at position d with isoleucine and at position a with leucine results in a peptide that tetramerizes. Peptides containing these substitutions are still referred to as leucine zipper domains. However, it should be pointed out that in a preferred embodiment leucine zippers capable of dimerizing proteins are used as RANKL fusion partners. Construction of a fusion RANKL-leucine zipper fusion protein may be performed in a similar manner as for GST-RANKL and AP-RANKL. See Example 18. In addition to bacteria, other suitable expression systems such as mammalian cells and insect cells may be used. One of ordinary skill in the art can easily make necessary adjustments in order to express a leucine zipper-RANKL fusion protein.

In an alternative embodiment, a RANKL derivative may be used to form oligomeric complexes. It has recently been discovered that a newly found TNF ligand family member TALL-1 (also known as BAFF, THANK, BLyS, and zTNF4) possesses the ability to oligomerize under physiological conditions (Liu et al., Cell, 108:383-394, 2002). Liu et al. have shown that the “flap” region, named so due to the length of the loop that forms the flap and allows it to extend from the molecule, mediates trimer-trimer ineractions and subsequent cluster formation. This flap region is unique to TALL-1 among TNF family members and is created by a surface DE loop (the loop that connects the strands D and E of TALL-1) that is longer than any DE loop of other TNF family proteins, which have been discovered so far. The oligmerization is thought to occur through a noncovalent interaction of the long DE loop with surrounding TALL-1 molecules, thereby resulting in the formation of large clusters. Since RANKL and TALL-1 are both TNF ligand family members and possess similar β-strand core structure, in accordance with the invention, RANKL is mutated to create a mutant RANKL molecule that oligomerizes spontaneously at physiological conditions. In one embodiment, modification of RANKL is designed so that its DE loop (amino acids 245-249 containing the amino acid sequence SIKIP) is substituted with the DE loop of TALL-1 (amino acid sequence KVHVFGDEL). See Example 19. To further recapitulate the oligomerization domains of TALL-1, the following amino acid changes may be made throughout the RANKL molecule: 168T→I, 187Y→L, 194K→F, 212F→Y, 252H→V, 279F→I, and 283R→E. See Example 20. The mutations can be introduced into RANKL by PCR-driven site-directed mutagenesis, using, for example, the QuickChange Multi-Site Directed Mutagenesis Kit (available from Stratagene). To determine the oligomerization potential of such modified RANKL molecule, one can use the same assays as for testing GST-RANKL, such as size-exclusion chromatography. One of ordinary skill in the art can make said mutations and test the structure and function of the mutated RANKL without undue experimentation.

In vitro or in vivo assays can be used to determine the efficacy of oligomeric RANKL complexes of the present invention in promoting bone formation in human and animal patients as taught by applicants. For in vitro binding assays, osteoblast-like cells can be used. Suitable osteoblast-like cells include, but are not limited to, primary marrow stromal cells, primary osteoblasts, ST-2 cells, C1 cells, ROS cells, and MC3T3-E1 cells. Many of the cell lines are available from American Type Culture Collection, Rockville, Md., and can be maintained in standard specified growth media. For in vitro functional assays, oligomeric complexes can be tested by culturing the cells with a range of concentrations of compounds and assessing markers or indicia of bone formation such as osteoblast activation, bone matrix deposition, calvarial thickness and bone nodule formation. See Example 2 below. In addition, osteoblast proliferation, expression of Collagen type I and/or expression of Cbfa1 may be used to assess bone formation. See Example 14 below.

Furthermore, a general protocol for treatment of osteoblasts with a compound is well established in the art. See, for instance, Wyatt et al., BMC Cell Biology, 2:14, 2001. A cell line of choice in this article was MC3T3-E1, which has been used as an in vitro model of osteoblastic differentiation and maturation. The treatment of cells, in this case with BMP-2, was performed in the following manner. The cells were plated at 5000/cm² in plastic 25 cm² culture flasks in α-MEM supplemented with 5% fetal bovine serum, 26 mM NaHCO₃, 2 mM glutamine, 100 u/ml penicillin, and 100 μg/ml streptomycin, and grown in humidified 5% CO₂/95% air at 37° C. Cells were passaged every 3-4 days after releasing with 0.002% pronase E in PBS. The cells in treatment groups were grown for 24 hours, then incubated with BMP-2 (50 ng/ml) dissolved in PBS containing 4 mM HCl and 0.1% bovine serum albumin (BSA) at 37° C. for 24 and 48 hours. Control groups received equal volumes of vehicles only.

Exemplary conditions for treatment of osteoblast cells or precursors with oligomers, such as GST-RANKL, are described below. Osteoblast precursor cells are incubated in the presence of vehicle, GST (a negative control), or increasing concentrations of purified oligomeric GST-RANKL (e.g. concentrations ranging from 1 ng/ml to 10 ng/ml). Bone morphogenetic protein (BMP)-2 is administered as a positive control. Test compositions are administered for a period of 12 hours only at the initiation of the culture or once at initiation and once three days later, again for a duration of 12 hours. It is to be noted that the conditions used will vary according to the cell lines and compound used, their respective amounts, and additional factors such as plating conditions and media composition. Such adjustments are readily determined by one skilled in this art.

Additionally, oligomeric RANKL compositions which enhance bone formation according to applicants methods may be evaluated in various animal models. See Examples 3-6 and descriptions below.

A commonly used assay is a neonatal mouse calvaria assay. Briefly, four days after birth, the front and parietal bones of ICR Swiss white mouse pups are removed by microdissection and split along the sagittal suture. The bones are then incubated in a specified medium, wherein the medium contains either test or control compounds. Following the incubation, the bones are removed from the media, and fixed in 10% buffered formalin for 24-48 hours, decalcified in 14% EDTA for 1 week, processed through graded alcohols, and embedded in paraffin wax. Three micron sections of the calvaria are prepared and assessed using histomorphometric analysis of bone formation or bone resorption. Bone changes are measured on sections cut 200 microns apart. Osteoblasts and osteoclasts are identified by their distinctive morphology.

In addition to this assay, the effect of compounds on murine calvarial bone growth can also be tested in vivo. In one such example of this screening assay, male ICR Swiss white mice, aged 4-6 weeks are employed, using 4-5 mice per group. Briefly, the test compound or the appropriate control is injected into subcutaneous tissue over the right calvaria of normal mice. The mice are sacrificed on day 14, and bone growth is measured by histomorphometric means. Bone samples are cleaned from adjacent tissues and fixed in 10% buffered formalin for 24-48 hours, decalcified in 14% EDTA for 1-3 weeks, processed through graded alcohols, and embedded in paraffin wax. Three to five micron sections of the calvaria are prepared, and representative sections are selected for histomorphometric assessment of the effects of bone formation and bone resorption. Sections are measured by using a camera lucida attachment to trace directly the microscopic image onto a digitizing plate. Bone changes are measured on sections cut 200 microns apart, over 4 adjacent 1×1 mm fields on both the injected and noninjected sides of calvaria. New bone is identified by its characteristic tinctorial features, and osteoclasts and osteoblasts are identified by their distinctive morphology. Histomorphometry software (OsteoMeasure, Osteometrix, Inc., Atlanta) can be used to process digitized input to determine cell counts and measure areas or perimeters.

Additional in vivo assays include dosing assays in intact animals, and dosing assays in acute ovariectomized (OVX) animals (prevention model), and assays in chronic OVX animals (treatment model). Prototypical dosing in intact animals may be accomplished by, for example, subcutaneous, intraperitoneal, transepithelial, or intravenous administration, and may be performed by injection, or other delivery techniques. The time period for administration of test compound may vary (for instance, 28 days as well as 35 days may be appropriate). As an example, in vivo transepithelial or subcutaneous dosing assays may be performed as described below.

In a typical study, 70 three-month-old female Sprague-Dawley rats are weight-matched and divided into seven groups, with ten animals in each group. This includes a baseline control group of animals sacrificed at the initiation of the study; a control group administered vehicle only; a PBS-treated control group; and a positive group administered a compound known to promote bone growth. Three dosage levels of the test compound are administered to the remaining groups. Test compound, PBS, and vehicle are administered subcutaneously once per day for 35 days. All animals are injected calcein nine days and two days before sacrifice (to ensure proper labeling of newly formed bone). Weekly body weights are determined. At the end of 35 days, the animals are weighed and bled by orbital or cardiac puncture. Serum calcium, phosphate, osteocalcin, and CBCs are determined. Both leg bones (femur and tibia) and lumbar vertabrae are removed, cleaned of adhering soft tissue, and stored in 70% ethanol or 10% formalin for evaluation, as performed by peripheral quantitative computed tomography (pQCT; Ferretti, J, Bone, 17: 353S-364S, 1995), dual energy X-ray absorptiometry (DEXA; Laval-Jeantet A. et al., Calcif Tissue Intl, 56:14-18, 1995, and Casez J. et al., Bone and Mineral, 26:61-68, 1994) and/or histomorphometry. The effect of test compounds on bone remodeling can thus be evaluated.

Test compounds can also be assayed in acute ovariectomized animals. Such assays may also include an estrogen-treated group as a control. An example of the test in these animals is briefly described below.

In a typical study, 80 three-month-old female Sprague-Dawley rats are weight-matched and divided into eight groups, with ten animals in each group. This includes a baseline control group of animals sacrificed at the initiation of the study; three control groups (sham OVX and vehicle only, OVX and vehicle only, and OVX and PBS only); and a control OVX group that is administered a compound known to enhance bone mass. Three dosage levels of the test compound are administered to remaining groups of OVX animals.

Since ovariectomy induces hyperphagia, all OVX animals are pair-fed with sham OVX animals throughout the 35 day study. Test compound, positive control compound, PBS or vehicle alone is administered transepithelially or subcutaneously once per day for 35 days. As an alternative, test compounds can be formulated in implantable pellets that are implanted for 35 days, or may be administered transepithelially, such as by nasal administration. All animals are injected with calcein at intervals determined empirically, including but not limited to nine days and two days before sacrifice. Weekly body weights are determined. At the end of the 35-day cycle, the animals blood and tissues are processed as described above.

Test compounds may also be assayed in chronic OVX animals. Briefly, 80 to 100 six month old female, Sprague-Dawley rats are subjected to sham surgery (sham OVX), or ovariectomy (OVX) at the beginning of the experiment, and 10 animals are sacrificed at the same time to serve as baseline controls. Body weights are monitored weekly. After approximately six weeks or more of bone depletion, 10 sham OVX and 10 OVX rats are randomly selected for sacrifice as depletion period controls. Of the remaining animals, 10 sham OVX and 10 OVX rats are used as placebo-treated controls. The remaining animals are treated with 3 to 5 doses of test compound for a period of 35 days. As a positive control, a group of OVX rats can be treated with a known anabolic agent in this model, such as PTH (Kimmel et al., Endocrinology, 132: 1577-1584, 1993). At the end of the experiment, the animals are sacrificed and femurs, tibiae, and lumbar vertebrae1 to 4 are excised and collected. The proximal left and right tibiae are used for pQCT measurements, cancellous bone mineral density (BMD), and histology, while the midshaft of each tibiae is subjected to cortical BMD or histology. The femurs are prepared for pQCT scanning of the midshaft prior to biomechanical testing. With respect to lumbar vertebrae (LV), LV2 are processed for BMD (pQCT may also be performed), LV3 are prepared for undecalcified bone histology, and LV4 are processed for mechanical testing.

In a further embodiment, applicants have discovered that the interaction between oligomeric RANKL and its receptor RANK on osteoblasts or osteoblast precursors results in prolonged intracellular activity of intracellular proteins. Mouse osteoblasts, when treated with GST-RANKL in vitro manifested activation, as characterized by the activation of NFκB and ERK intracellular signal pathways. As noted by the applicants, the time course of intracellular protein activity, especially ERK activity is different from that observed in osteoclast precursors, which also express RANK on the surface. In osteoclast precursors, ERK activity peaks 5-15 minutes after RANK/GST-RANKL interaction, and returns to basal levels after 15-30 minutes. In contrast, the ERK activity in osteoblasts peaks at 10 minutes after the same interaction, and is still above the basal level after 60 minutes. The prolongation of the time course is even more prominent in osteoblast precursor cells, wherein the demonstrated activity of ERK had not reached its maximum even 60 minutes after the RANK/oligomeric GST-RANKL interaction. Besides the different time course of ERK activity, osteoblasts and osteoblast precursor cells also exhibit prolonged activity of kinases such as IKK, P13 kinase, Akt, p38 and JNK. This osteoblast-related activity contrasts with GST-RANKL interaction with RANK on osteoclasts, which results in short-lived activity of MAP kinases and bone resorption. While not being bound to a particular theory, it therefore appears that the prolonged activity of kinases observed in osteoblasts following oligomeric GST-RANKL stimulation plays a role in the anabolic bone processes.

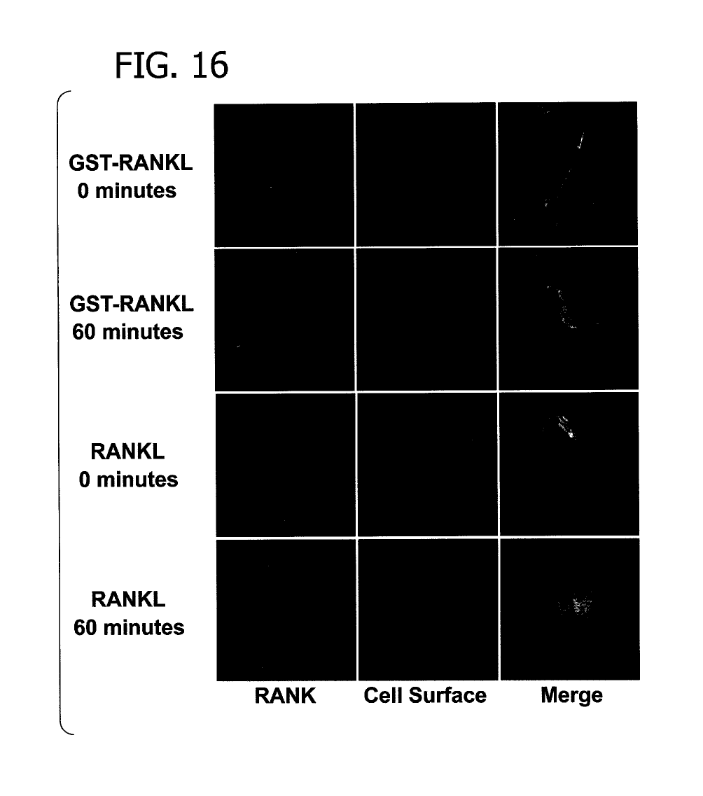

It is known that TNF family cytokine-induced intracellular signaling is attenuated by internalization of the receptor-ligand complex (see, e.g., Higuchi, M and Aggarwal, B. B., J. Immunol., 152:3550-3558, 1994). Applicants, therefore believe that oligomeric complexes comprising RANKL are not internalized as promptly as RANKL trimers, thus allowing for a longer interaction with the receptor and prolonged intracellular signaling. See FIG. 16 and Example 13.

Accordingly, osteogenic compounds capable of enhancing activity of one or more intracellular proteins in osteoblasts or osteoblast precursors, wherein such activity is indicative of bone formation, may be used in the methods of the present invention Activated intracellular proteins include but are not limited to kinases. Preferably, the kinases comprise ERK1/2, JNK, P13 kinase, IKK, Akt, and p38, and even more preferably, the kinases are ERK1/2. Other intracellular proteins include IKB-α and IKB-β.

In another preferred embodiment, the activity comprises phosphorylation of one or more intracellular proteins, and more preferably of kinases. For the MAP kinase family, full activation requires dual phosphorylation on tyrosine and threonine residues separated by a glutamate residue (known as TEY motif, where T is threonine, E is glutamic acid, and Y is tyrosine) by a single upstream kinase known as MAP kinase kinase (MKK). The requirement for dual phosphorylation ensures that MAP kinases are specifically activated by the action of MKK.

Any of the assays available in the art for determining whether a kinase has been phosphorylated may be used. Preferably, such assays include Western blots or kinase assays.

A Western blot can be generally performed as follows. Once the cell lysates are generated, the intracellular proteins are separated on the basis of size by utilizing SDS-PAGE (sodium dodecyl sulfate-polyacrylamide gel electrophoresis). The separated proteins are transferred by electroblotting to a suitable membrane (such as nitrocellulose or polyvinylidene flouride) to which they adhere. The membrane is washed to reduce non-specific signals, and then probed with an antibody which recognizes only the specific amino acid which has been phosphorylated as a result of RANK signaling. After further washing, which removes excess antibody, a second antibody, which recognizes the first antibody (bound to specifically-phosphorylated proteins on the membrane) and contains a reporter moiety is applied to the membrane. The addition of a developing agent, which interacts with a reporter moiety on the second antibody results in visualization of the bands.

A kinase assay, for example for ERK1/2, can be performed by utilizing a known substrate for this kinase such as p90 ribosomal S6 protein kinase (RSK). Briefly, by way of example, treated osteoblasts are washed in ice-cold PBS, e.g., three times, and extracted with lysis buffer in order to obtain cell lysates. Supernatants obtained after microcentifugation of cell lysates are incubated with goat anti-RSK2 antibody (1:200) together with protein G-Sepharose at 4° C. overnight. The beads are collected by microcentrifugation, washed twice with lysis buffer, followed by kinase buffer. RSK2 phosphotransferase activity in the beads is measured by using S6 kinase assay kit and [γ-³²P]ATP according to the protocols provided by the manufacturer (Upstate Biotechnology, Inc).

An additional assay that can be applied to determine activation of osteoblasts is an electrophoretic mobility gel shift assay (EMSA). This assay monitors nuclear translocation of a transcription factor complex (such as NFκB following activation of osteoblasts with GST-RANKL). Briefly, an EMSA may be conducted as follows. Nuclei of treated osteoblasts are isolated and their extracts generated. The nuclear proteins are then incubated with a specific oligonucleotide probe that has been labeled with ³²P orthophosphate. After an appropriate time, the putative protein-DNA complexes are separated on a PAGE gel (no SDS present), which is dried and exposed to an X-ray film. If a specific complex has formed (in this case a complex of NFκB proteins with a specific DNA sequence) a band will be visible on the developed film. Typically, appropriate controls are run in parallel with the experimental sample(s) in order to ensure that the band is specific for activated osteoblasts. For detailed procedures on Western blotting, kinase assays, and EMSA, see for example Lai et al., Journal of Biological Chemistry, 276(17):14443-14450, Apr. 27, 2001.

The activation in osteoblasts can be detected up to at least 60 minutes following the incubation of said cells with oligomers, such as GST-RANKL. In osteoblast precursor cells, the activation peaks after 5-10 minutes, and can be detected for up to at least 60 minutes. Accordingly, the activity of one or more intracellular proteins may be detected for at least about 30 minutes after the incubation of the osteogenic compound with osteoblasts or osteoblast precursors. In a preferred embodiment, the activity is detected for at least about 40 minutes, and more preferably for at least about 60 minutes after said incubation. In another preferred embodiment, the intracellular proteins whose activity is detected for at least about 30 minutes are kinases, and more preferably, the kinases are ERK1/2.

To confirm that a compound that activates osteoblasts and/or stimulates differentiation of osteoblast precursors can enhance anabolic bone processes, such compound can be tested in a bone formation assay, wherein an increase in bone mass over the increase in background bone mass designates a compound as having osteogenic activity. There are multiple bone formation assays that can be used successfully to screen potential osteogenic compounds of this invention. For example, cell-based assays for osteoblast differentiation and function, based on measuring collagen levels and alkaline phosphatase activity may be used. These assays are well known in the art and easily performed by a skilled artisan. Furthermore, multiple in vitro and in vivo bone formation assays have been described in above sections. It should be noted that in vitro assays may be performed with either osteoblasts or osteoblast precursors since both cell types exhibit prolonged activity of the same kinases following stimulation with anabolic forms of RANKL, such as GST-RANKL.

In cases when the intracellular activation assays and bone formation assays are performed with a library of compounds, it may be necessary to positively identify a compound that has shown to be osteogenic. There are multiple ways to determine the identity of the compound. One process involves mass spectrometry, available from Neogenesis (http://www.neogenesis.com). Neogenesis' ALIS (automated ligand identification system) spectral search engine and data analysis software allow for a highly specific identification of a ligand structure based on the exact mass of the ligand. One skilled in the art may also perform mass spectrometry experiments to determine the identity of the compound.

In another embodiment, osteogenic compounds capable of inactivating one or more phosphatases in osteoblasts or osteoblast precursors, wherein said inactivation is indicative of bone formation may be used in the methods of the present invention. In one preferred embodiment, the phosphatases inhibit the kinases involved in osteogenesis, including p38, ERKs, JNK, IKK, and Akt. More preferably, the phosphatases are MAPK specific or Akt specific, and even more preferably they are ERK1/2 specific. While not being bound to a particular theory, this method is feasible for this purpose due to the fact that a kinase activity is tightly regulated by its corresponding phosphatase. In case of ERK1/2, the phosphatase is known as the mitogen activated protein kinase phosphatase-3 (MKP-3). This phosphatase belongs to a family of dual specificity phosphatases, which are responsible for the removal of phosphate groups from the threonine and tyrosine residues on their corresponding kinases (Camps et al., FASEB J., 14, pp.6-16, 1999). The prompt removal of phosphate groups by phosphatases ensures that kinase activation is short-lived and that the level of phosphorylation is low in a resting cell. However, in order for the phosphatase to be active and remove phosphate groups, it also needs to be phosphorylated. Therefore, inhibition of phosphatase activity results in activation or prolongation of ERK1/2 activity.

One method of determining the ability of an osteogenic compound to inactivate phosphatases in osteoblasts/osteoblast precursors involves initially activating osteoblasts/osteoblast precursors with a substance known to activate these cells, such as GST-RANKL or BMP-2 (bone morphogenetic protein 2). This leads to activation of phosphatases, at which point osteoblasts/osteoblast precursors are treated with a test compound and cell lysates are obtained. The ability of the test compound to dephosphorylate (inactivate) phosphatase(s) is determined by performing Western blots or kinase assays. See above. For additional details on assessing phosphatase activity, see Muda et al., J Biol Chem., 273:9323-9329, 1998, and Camps et al., Science 280:1262-1265, 1998. If the compound is determined to possess phosphatase inhibitory activity, it can further be tested in one of the bone formation assays to determine its osteogenic activity. These assays were also described above.

Pharmaceutical Compositions and Methods

In a preferred embodiment of the invention, a method of preventing or inhibiting bone loss or of enhancing bone formation is provided by administering 1) oligomeric complexes of one or more of RANKL, a RANKL fusion protein, analog, derivative, or mimic, 2) osteogenic compounds capable of enhancing activity of intracellular proteins in osteoblasts or osteoblast precursors, wherein said activity is indicative of bone formation, or 3) osteogenic compounds capable of inactivating intracellular proteins in osteoblasts or osteoblast precursors, wherein said inactivation is indicative of bone formation. The bone forming compositions of the present invention may be utilized by providing an effective amount of such compositions to a patient in need thereof. In one preferred embodiment, such compositions are used to treat conditions selected from the group consisting of: osteoporosis, juvenile osteoporosis, osteogenesis imperfecta, hypercalcemia, hyperparathyroidism, osteomalacia, osteohalisteresis, osteolytic bone disease, osteonecrosis, Paget's disease, rheumatoid arthritis, inflammatory arthritis, osteomyelitis, corticosteroid treatment, periodontal disease, skeletal metastasis, cancer, age-related bone loss, osteopenia, and degenerate joint disease.

For use for treatment of animal subjects, the compounds of the invention can be formulated as pharmaceutical or veterinary compositions. Depending on the subject to be treated, the mode of administration, and the type of treatment desired, e.g., prevention, prophylaxis, therapy; the compounds are formulated in ways consonant with these parameters. A summary of such techniques is found in Remington's Pharmaceutical Sciences, latest edition, Mack Publishing Co., Easton, Pa.

The administration of RANKL-comprising oligomers or osteogenic compounds of the present invention may be pharmacokinetically and pharmacodynamically controlled by calibrating various parameters of administration, including the frequency, dosage, duration mode and route of administration. Thus, in one embodiment bone mass formation is achieved by administering anabolic compositions such as an oligomeric complex of one or more of RANKL, a RANKL fusion protein, analog, derivative or mimic in a non-continuous, intermittent manner, such as by daily injection and/or ingestion. Generally, any osteogenic compound as described herein may be administered intermittently to achieve the same affect. Variations in the dosage, duration and mode of administration may also be manipulated to produce the activity required.

For administration to animal or human subjects, the dosage of the compounds of the invention is typically 0.01-100 mg/kg. However, dosage levels are highly dependent on the nature of the disease or situation, the condition of the patient, the judgment of the practitioner, and the frequency and mode of administration. If the oral route is employed, the absorption of the substance will be a factor effecting bioavailabiity. A low absorption will have the effect that in the gastro-intestinal tract higher concentrations, and thus higher dosages, will be necessary.

It will be understood that the appropriate dosage of the substance should suitably be assessed by performing animal model tests, wherein the effective dose level (e.g. ED₅₀) and the toxic dose level (e.g. TD₅₀) as well as the lethal dose level (e.g. LD₅₀ or LD₁₀) are established in suitable and acceptable animal models. Further, if a substance has proven efficient in such animal tests, controlled clinical trials should be performed.

In general, for use in treatment, the compositions of the invention may be used alone or in combination with other compositions for the treatment of bone loss. Such compositions include anti-resorptives such as a bisphosphonate, a calcitonin, a calcitriol, an estrogen, SERM's and a calcium source, or a supplemental bone formation agent like parathyroid hormone or its derivative, a bone morphogenetic protein, osteogenin, NaF, or a statin. See U.S. Pat. No. 6,080,779 incorporated herein by reference. Depending on the mode of administration, the compounds will be formulated into suitable compositions.

Formulations may be prepared in a manner suitable for systemic administration or for topical or local administration. Systemic formulations include, but are not limited to those designed for injection (e.g., intramuscular, intravenous or subcutaneous injection) or may be prepared for transdermal, transmucosal, or oral administration. The formulation will generally include a diluent as well as, in some cases, adjuvants, buffers, preservatives and the like.

For transepithelial administration, penetrants appropriate to the barrier to be permeated are used in the formulation. Such penetrants are generally known in the art. For oral administration, the compounds can be administered also in liposomal compositions or as microemulsions. Suitable forms include syrups, capsules, tablets, as is understood in the art. For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

RANKL-comprising oligomers and osteogenic compounds described herein also may be administered locally to sites in patients, both human and other vertebrates, such as domestic animals, rodents and livestock, where bone formation and growth are desired using a variety of techniques known to those skilled in the art. For example, these may include sprays, lotions, gels or other vehicles such as alcohols, polyglycols, esters, oils and silicones. Such local applications include, for example, at a site of a bone fracture or defect to repair or replace damaged bone. Additionally, oligomeric complexes and osteogenic compounds of the present invention may be administered e.g., in a suitable carrier, at a junction of an autograft, allograft or prosthesis and native bone to assist in binding of the graft or prosthesis to the native bone.

Pharmaceutically acceptable excipients include, but are not limited to, physiological saline, Ringer's, tocopherol, phosphate solution or buffer, buffered saline, and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective.

The following examples illustrate the invention, but are not to be taken as limiting the various aspects of the invention so illustrated.

### EXAMPLES

### Example 1

Expression of RANKL as a GST-RANKL Fusion Protein.

cDNA encoding murine RANKL residues 158-316 was cloned into pGEX-4T-1 (Amersham; GenBank Accession No. U13853—see National Library of Medicine listing at http://ncbi.nlm.nih.gov under nucleic acids.) downstream of glutathione S-transferase using the SalI and NotI restriction endonucleases. Following IPTG-mediated (0.05 mM) induction of protein expression in BL21 (DE3) Escherischia coli (Invitrogen), cells were triturated into a lysis buffer comprising 150 mM NaCl, 20 mM Tris-HCl pH 8.0, and 1 mM EDTA. Lysates were incubated with glutathione sepharose (Amersham) for affinity purification of the GST-RANKL fusion protein, followed by excessive washing with buffer comprising 150 mM NaCl and 20 mM Tris-HCl pH 8.0. Following competitive elution (10 mM reduced glutathione) from the affinity column, the isolated protein was then subjected to ion exchange chromatography, eluted with a salt gradient ranging from 0-500 mM NaCl, and dialyzed against physiologic salt and pH. Purified GST-RANKL was then assayed for endotoxin contamination by limulus amoebocyte lysate assay, and quantitated for bioactivity by an in vitro osteoclastogenesis readout.

Under conditions replicating the physiological milieu, GST-RANKL forms large oligomeric complexes, as demonstrated by size exclusion chromatography. See FIG. 2. The majority of the protein, as determined by the area under the curve in FIG. 2, exists as oligomeric complexes of GST-RANKL.

### Example 2

Ex vivo Stimulation of Bone Formation in Whole Calvarial Organ Culture.

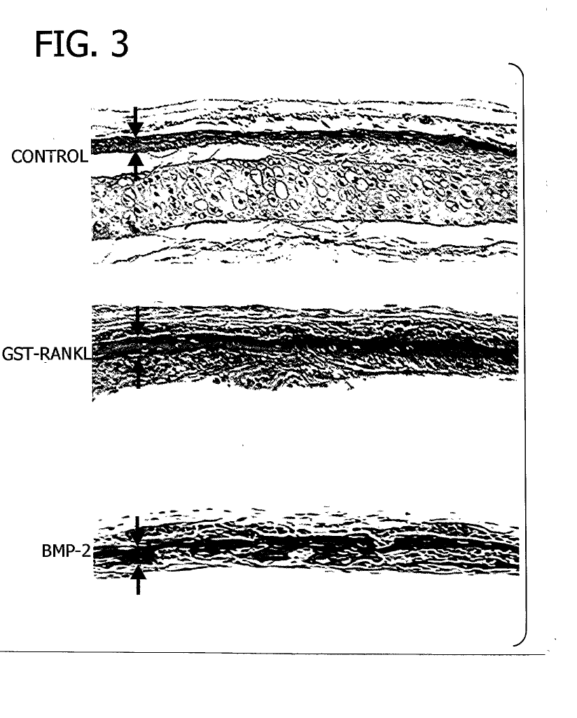

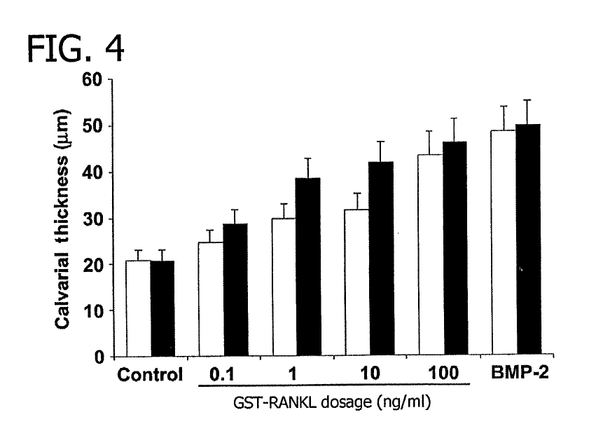

An assay for bone formation was carried out as described in U.S. Pat. No. 6,080,779 col. 10, 11. 29-55 incorporated herein by reference. Neo-natal mouse calvariae were placed in organ culture in the presence of vehicle, GST (a negative control), or increasing concentrations of purified GST-RANKL obtained as outlined in Example 1. Bone morphogenetic protein (BMP)-2 was administered as a positive control. Test compositions were administered for a period of 12 hours only at the initiation of the culture (1×) or once at initiation and once three days later, again for a duration of 12 hours (2×). After seven days, calvarial thickness was determined histomorphometrically and compared among the various control and experimental groups to assess bone formation. Briefly, calvarial bones were removed from the incubation medium, fixed in 10% neutral buffered formalin for 12 hours, decalcified in 14% EDTA for 3 days, dehydrated through graded alcohols, and embedded in paraffin for histological sectioning. Calvaria were sectioned coronally through the central portion of the parietal bone, perpendicular to the sagittal suture. Representative coronal sections of comparable anatomic position were subjected to histomorphometric assessment (OsteoMeasure, Osteometrics Inc., Atlanta, Ga.) of calvarial thickness. See FIG. 3. GST-RANKL induced a dose-dependent increase in cavarial thickness when administered 1× or 2×. See FIG. 4. At the highest doses tested (100 ng/ml) calvarial thickness had doubled.

### Example 3

In vivo Stimulation of Bone Formation in Mice.

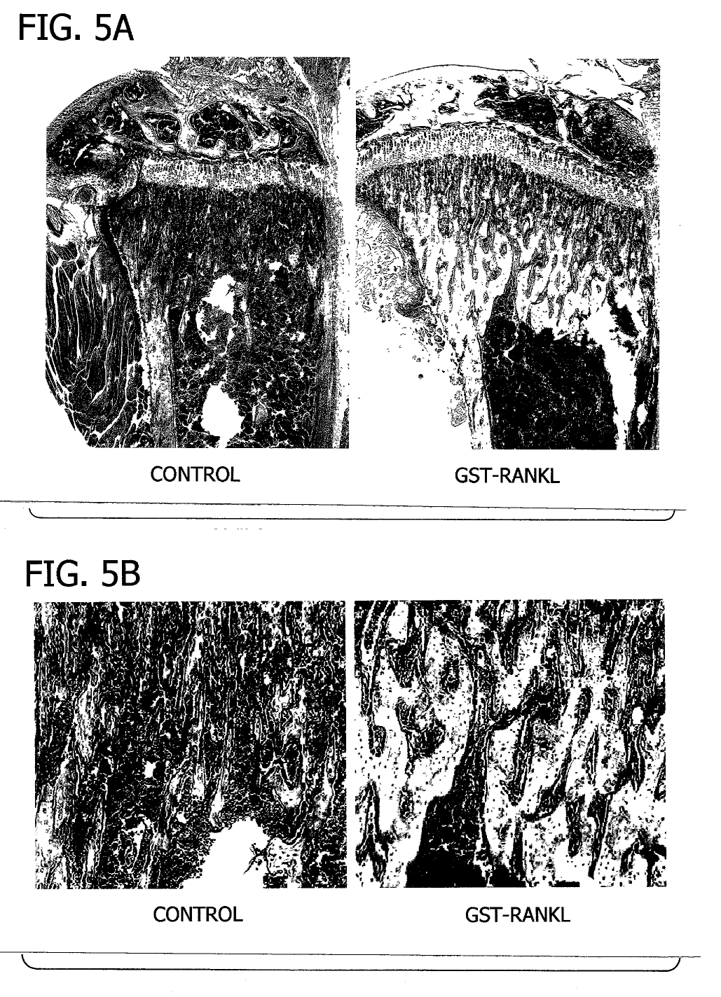

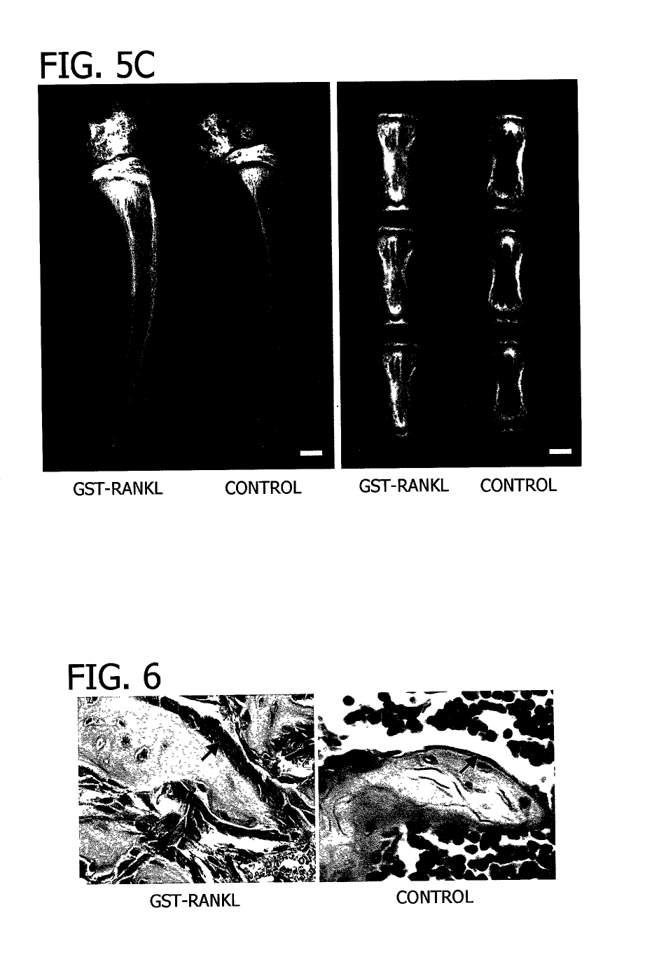

Mice, C3H/HeN (Harlan, Indianapolis, Ind.) were administered 100 micrograms GST (control) or 100 micrograms GST-RANKL as obtained in Example 1, subcutaneously, once a day for nine days. Histological examination of tibia reveals a marked increase in bone mass and a net increase in the numbers of activated osteoblasts in GST-RANKL-treated as compared to control mice. See FIGS. 5(a) and 5(b), taken at low power and high power magnification, respectively. The figures revealed a marked increase in cortical thickness and augmentation of the trabecular architecture of the primary spongiosa, relative to control animals receiving GST.

Dual-energy X-ray absorptiometry (DEXA) analysis of GST or GST-RANKL administered mice was also conducted using standard procedures. Results (see FIG. 5(c) show a significant increase in bone mineral density of GST-RANKL compared to control.

### Example 4

In vivo Activation of Osteoblasts.

Mice C3H/HeN (Harlan, Indianapolis, Ind.) were administered GST (control) or GST-RANKL, following the procedure set forth in Example 3. Histological examination of tibia at high magnification revealed a marked activation of osteoblasts in GST-RANKL-treated as compared to control mice. Quiescent osteoblasts are evident in control animals as thin bone-lining cells, whereas activated osteoblasts are evident in GST-RANKL-treated animals as plump, cuboidal cells along the bone surface. See FIG. 6.

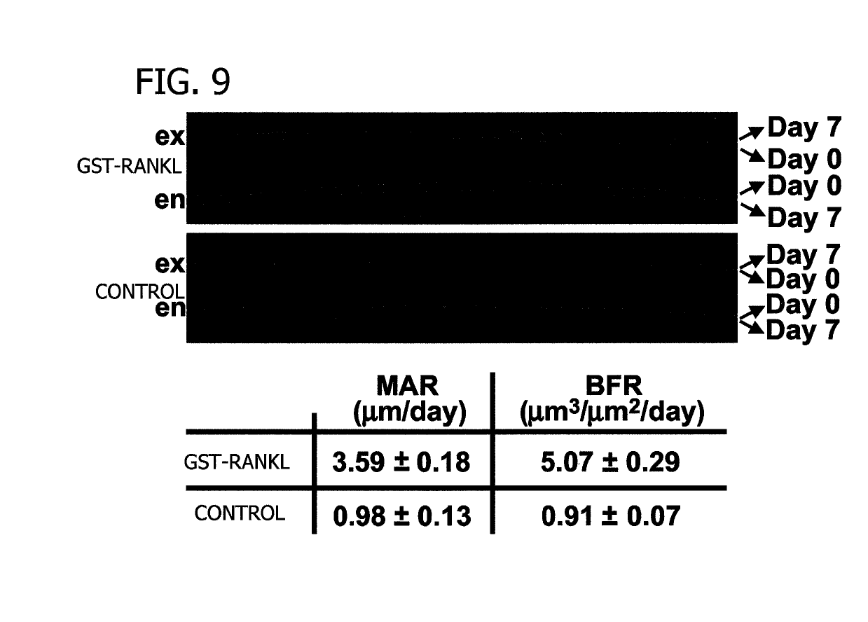

Measurement of the rate of bone formation during in vivo administration of GST-RANKL, versus GST control, was accomplished by intraperitoneal administration of 20 mg/kg calcein in 2% NaHCO₃ seven and two days before euthanasia to allow incorporation of two fluorescent labels into mineralizing bone matrix. Following dissection, calvaria were fixed in 70% EtOH and embedded in polymethyl methacrylate for histological sectioning. Shown in FIG. 9 are fluorescent micrographs of coronal sections of the parietal bone taken mid-way between the coronal and lambdoidal sutures, with the external surface of the calvarium oriented upwards on the figure and the internal surface oriented downwards. The amount of bone synthesized during the five day period is that encompassed within the two sets of parallel fluorescent bands. While the magnitude of bone formation in control animals receiving only GST is insufficient to produce distinctly separated double labels, there is clear deposition of bone during the five days between the first and second labels in GST-RANKL-treated animals.

### Example 5

Administration of GST-RANKL Stimulates Osteoblast Proliferation Without Substantially Affecting Osteoclastogenesis.

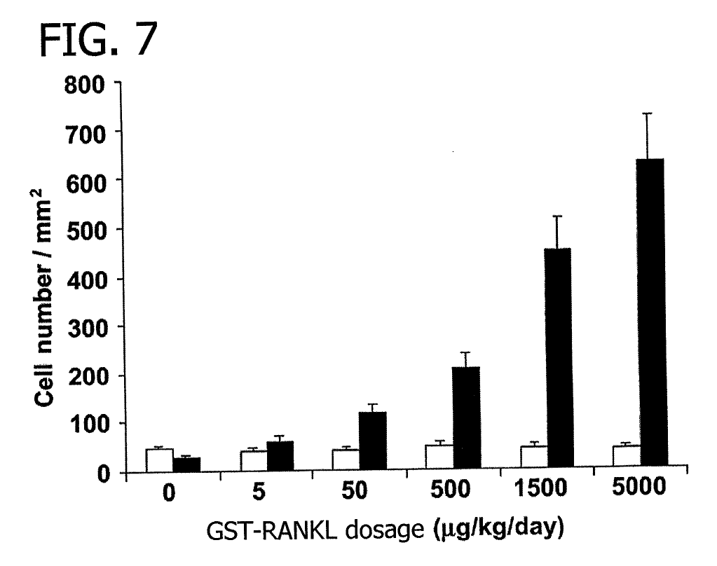

Purified GST-RANKL fusion product was administered subcutaneously to mice C3H/HeN (Harlan, Indianapolis, Ind.), in increasing dosages of 5, 50, 500, 1,500, 5,000 μg/kg, once a day, for 7 days. GST in moles equivalent to the highest dosage of RANKL served as a negative control. The mice were sacrificed and long bones were fixed, decalcified and stained for tartrate resistant acid phosphatase (TRAP) activity. TRAP activity is a specific phenotypic marker of the osteoclast in the context of bone. The number of activated osteoblasts and osteoclasts, per mm trabecular bone surface was histomorphometrically quantitated. As seen in FIG. 7, GST-RANKL administered in an intermittent fashion (namely, by daily injection), resulted in a dose-dependent increase in activated osteoblast, but not osteoclast number. GST had no noticeable impact on either osteoblasts or osteoclasts.

### Example 6

Enhancement of Osteoblast Precursor Differentiation as Evidenced by ex vivo Bone Nodule Formulation.

Equal numbers of marrow cells from GST-RANKL (100 μg) and GST treated mice, as discussed in Example 3, were placed in osteoblastogenic conditions for 28 days to determine if the number of osteoblasts and their committed precursors capable of forming bone were increased. After the 28 days, the cells were stained with Alizarin red to identify mineralized bone nodules and Hematoxylin to identify colony forming units.

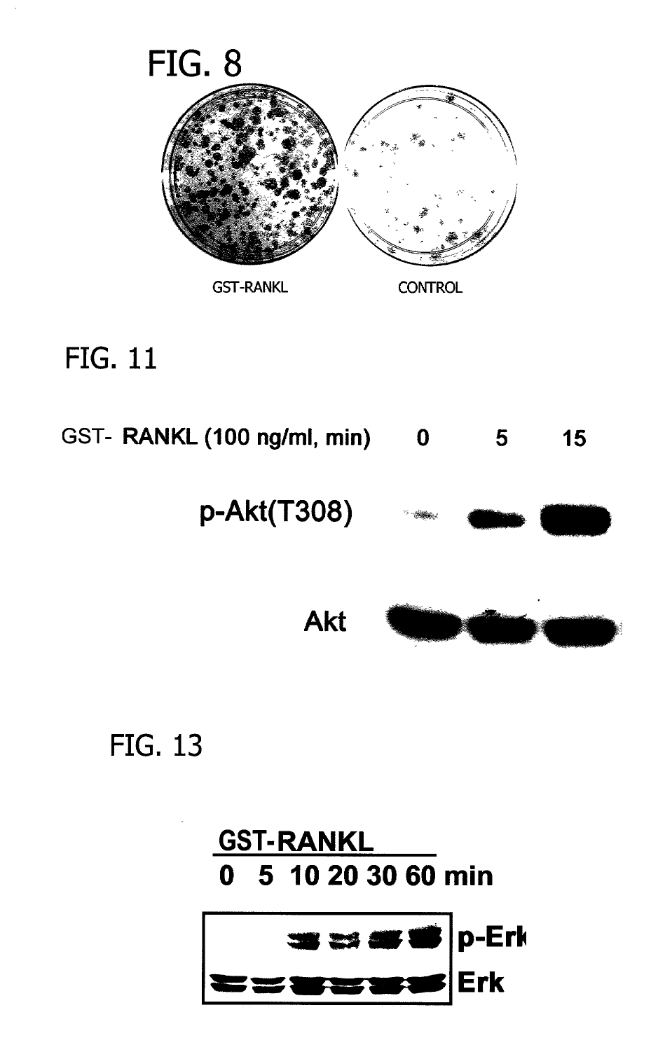

Marrow cells derived from GST-RANKL treated mice generated substantially more mineralized bone nodules than did their GST administered counterparts (See FIG. 8).

### Example 7

GST-RANKL Rapidly Activates MAP Kinases in Murine Osteoclast Precursors.

Wild type C57BL/6 mice were purchased from Harlan Industries (Indianapolis, Ind.). For the isolation of osteoclast precursors, bone marrow macrophages (BMMs) were isolated from whole bone marrow of four to six week old mice and incubated in tissue culture dishes at 37° C. in 5% CO₂. After 24 hours in culture, the non-adherent cells were collected and layered on a Ficoll Hypaque gradient and the cells at the gradient interface were collected. Cells were replated at 65,000/cm² in α-minimal essential medium, supplemented with 10% heat inactivated fetal bovine serum, at 37° C. in 5% CO₂ in the presence of recombinant mouse M-CSF (10 ng/ml). Cells were treated with GST-RANKL on day 4 or 5. In the experiments addressing the activity of Akt, the cells were cultured in serum and M-CSF free medium for 24 hours prior to GST-RANKL stimulation.

Immunoblotting (Western blotting) of osteoclast precursors was performed according to the following instructions. Cytokine-treated or control monolayers of BMMs were washed twice with ice-cold PBS. Cells were lysed in the buffer containing 20 mM Tris (pH 7.5), 150 mM NaCl, 1 mM EDTA, 1 mM EGTA, 1% Triton X-100, 2.5 mM sodium pyrophoshate, 1 mM β-glycerophosphate, 1 mM Na₃PO₄, 1 mM NaF, and 1× protease inhibitor cocktail. Fifty μg of cell lysates were boiled in the presence of SDS sample buffer (0.5 M Tris-HCl, pH 6.8, 10% w/v SDS, 10% glycerol, 0.05% w/v bromphenol blue) for 5 minutes and separated on SDS-PAGE, using 8% gels. Proteins were transferred to nitrocellulose membranes using a semi-dry blotter (Bio-Rad, Richmond, Calif.) and incubated in blocking solution (5% non-fat dry milk in tris-buffered saline containing 0.1% Tween 20) for 1 hour to reduce nonspecific binding. Membranes were then exposed to primary antibodies overnight at 4° C., washed three times, and incubated with secondary goat anti-mouse or rabbit IgG horseradish peroxidase-conjugated antibody for 1 hour. Membranes were washed extensively, and enhanced chemiluminiscence detection assay was performed following the manufacturer's directions (Amersham).

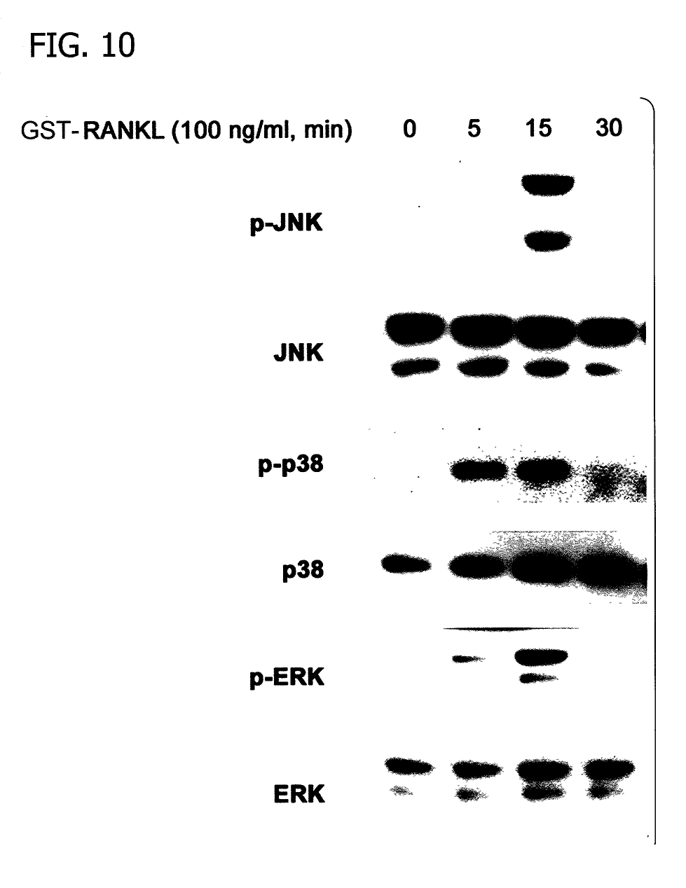

The results of the immunoblotting assay are depicted in FIG. 10. As can be seen from this figure, the total cellular amounts of JNK, p38, and ERK did not change significantly at any point of the assay. The phosphorylation (activation) of ERK and p38 was detected 5 minutes following the GST-RANKL stimulation, peaked at 10 minutes after RANK/GST-RANKL interaction, and was undetectable 30 minutes after the interaction. JNK was phosphorylated 15 minutes after the GST-RANKL stimulation, however the protein was also rapidly dephosphorylated so that by 30 minutes following GST-RANKL stimulation, phosphorylated forms of JNK were undetectable. The data indicated transient and short-lived activity of ERK, JNK, and p38 in murine osteoclast precursors following the GST-RANKL stimulation.

### Example 8

GST-RANKL Rapidly Activates Akt in Murine Osteoclast Precursors.

Osteoclast precursors were isolated, maintained, and manipulated as described in Example 7. Immnublotting protocol was also the same as in Example 7, except that a primary antibody was specific for phospho-Akt, obtained from Cell Signaling.

FIG. 11 shows that there was a detectable phosphorylation of Akt at the time of GST-RANKL stimulation, indicating rapid activation of this protein. Akt is a substrate for P13 kinase, and in its active state is involved in anti-apoptotic signaling. Akt activity increased with time, i.e. the number of phosphorylated Akt molecules in osteoclast precursors increased with time. Thus, the activity of Akt was greater at 5 minutes than at 0 minutes, and it peaked at 15 minutes following GST-RANKL stimulation.

### Example 9

GST-RANKL-Induced Activity of MAP Kinases is Prolonged in Murine Osteoblasts.

Primary osteoblasts were isolated from neonatal murine calvaria by sequential enzymatic digestion. Briefly, calvaria were minced and incubated at room temperature for 20 minutes with gentle shaking in an enzymatic solution containing 0.1% collagenase, 0.05% trypsin, and 4 mM NA₂EDTA in calcium- and magnesium-free phosphate buffered saline (PBS). This procedure was repeated to yield a total of six digests. The cells isolated from the last four to six digests were cultured in MEM containing 15% FBS, 50 μM ascorbic acid, and 10 mM β-glycerophosphate. Cells were maintained at 37° C. in a humidified atmosphere containing 6% CO₂, with daily replenishment of media and cytokines.

Following cytokine treatment at the indicated times and dosages, cells were lysed in RIPA buffer containing 10 mM Tris-HCl pH 7.4, 150 mM NaCl, 1% Nonidet P-40, 0.2% sodium deoxycholate, and 1 mM EDTA, with 1 mM Na₃PO₄, 1 mM NaF, and 1× protease inhibitor cocktail added immediately prior to use. Protein concentration was quantitated and standardized by Micro BCA Protein Assay (Pierce). Lysates were denatured by heat in Laemmli buffer, resolved by SDS-PAGE, and transferred onto nitrocellulose. Levels of total and phosphorylated ERK, JNK, p38, Akt, and IkBα were determined using primary and secondary antibodies according to the manufacturer's established protocols, with conventional chemiluminiscent detection. Membranes were stripped between hybridizations in PBS containing 10 μM β-mercaptoethanol and 2% SDS.

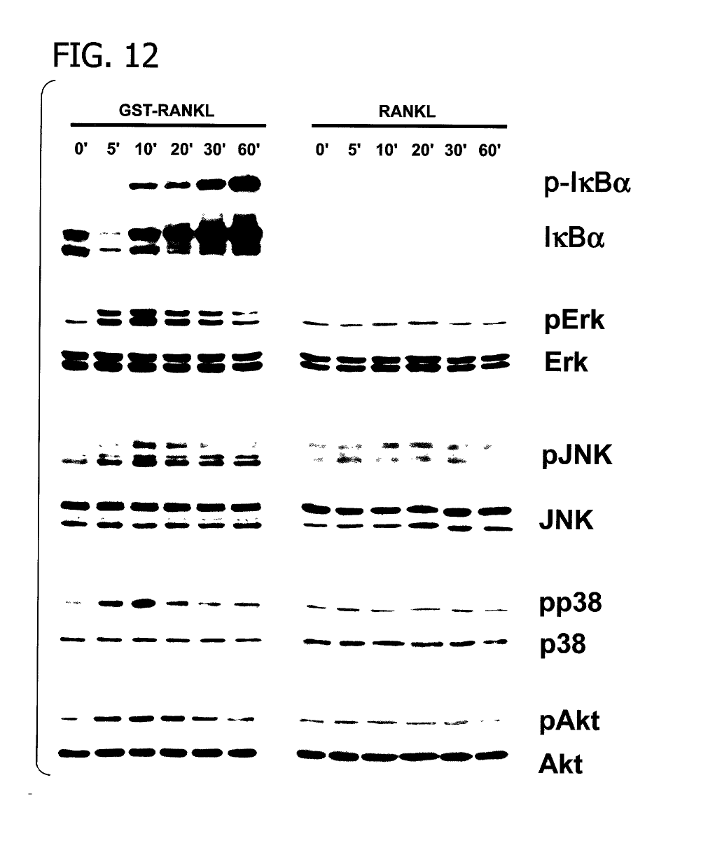

The results of the immunoblot assay measuring the activity of MAP kinases following GST-RANKL or equimolar RANKL stimulation are shown in FIG. 12. GST-RANKL stimulation was performed as described in Example 7. The kinases whose phosphorylation was measured include ERK, JNK, p38, and Akt. Again, as seen in osteoclast precursors, the amount of total protein did not significantly change in the cell at any time points. However, all of the kinases tested exhibited prolonged activity in osteoblasts. Both ERKs were activated by 5 minutes after GST-RANKL stimulation, and their activity could be detected at 60 minutes following the stimulation. The activity of JNK, p38, and Akt was detectable at the time of GST-RANKL stimulation, and could be detected for at least 60 minutes following the stimulation. In addition, phosphorylation of IkBα was detected 10 minutes after the stimulation and it increased until the end of the assay (60 minutes), indicating increased translocation of NFkB into the nucleus. The data suggest that the pattern of MAP kinase activity is different from the activity of the same kinases in osteoclasts. The prolonged activity observed in osteoblasts seems to play a role in accelerated anabolic bone processes. In addition, RANKL treatment was not able to induce prolonged activity of kinases as was seen with GST-RANKL.

### Example 10

GST-RANKL-Induced ERk1/2 Activity is Prolonged in Murine Osteoblast Precursors.

Osteoblast precursors were isolated and maintained according to the procedures set forth in Example 9. The immunoblotting was performed in the same manner as immunoblotting in Example 9.

As observed in FIG. 13, ERK activity in osteoblast precursors was prolonged and it increased with time. Whereas in osteoblasts the activity was prolonged but did not change significantly over time, ERK activity in osteoblast precursors was first detected at 10 minutes following GST-RANKL stimulation, and it increased up to 60 minutes following the activation, which was the length of time for which the assay was performed.

### Example 11

AP Activity Following GST-RANKL Exposure in Osteoblasts.

Primary calvarial osteoblasts were cultured in MEM containing 15% FBS, 50 μM ascorbic acid, and 10 mM β-glycerophosphate. Cells were maintained at 37° C., with daily replenishment of media and cytokines. Osteoblast alkaline phosphatase (AP) activity, a direct measure of osteoblast differentiation and function, was quantitated by addition of a colorimetric substrate, 5.5 mM p-nitrophenyl phosphate. The cells were then exposed to GST-RANKL, administered in different regimens. Pulsatile exposure to 50 ng/ml GST-RANKL was provided as 1, 3, 6, 8, or 24 hours of total exposure per 48-hour treatment window. After 4 such 48-hour treatments, AP activity was quantitated (±S.D.) and normalized to total protein levels.

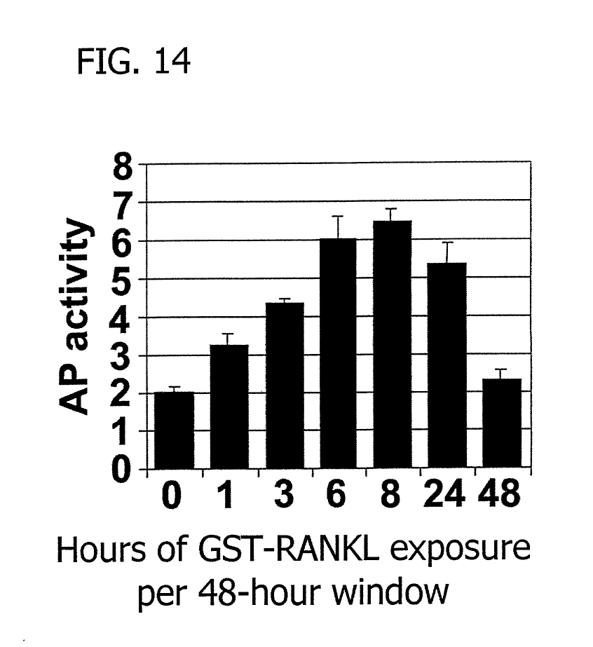

As can be seen from FIG. 14, the maximum anabolic effect was observed when GST-RANKL exposure was provided for an 8-hour treatment window, once every 48 hours. Thus, GST-RANKL induced increase in AP activity when administered in an intermittent fashion.

### Example 12

Oligomerization of GST-RANKL.

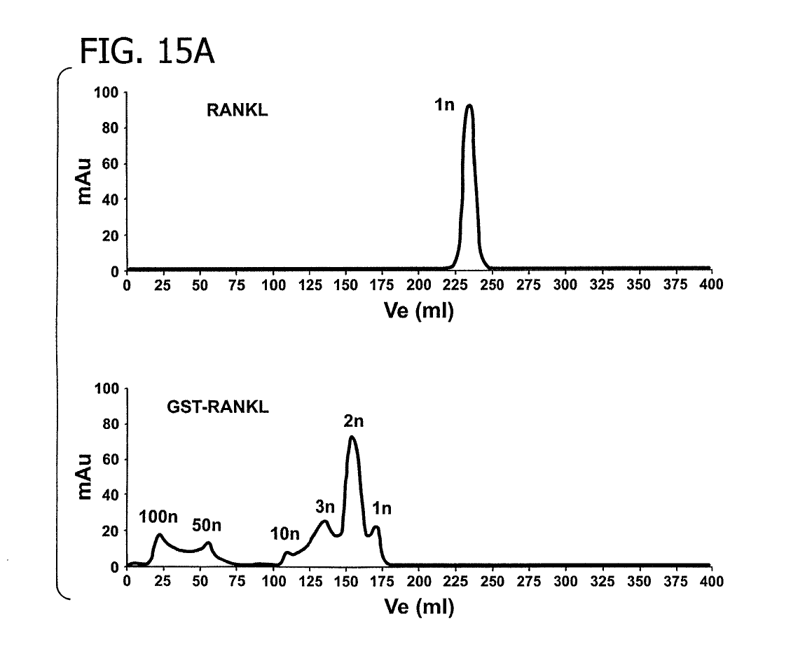

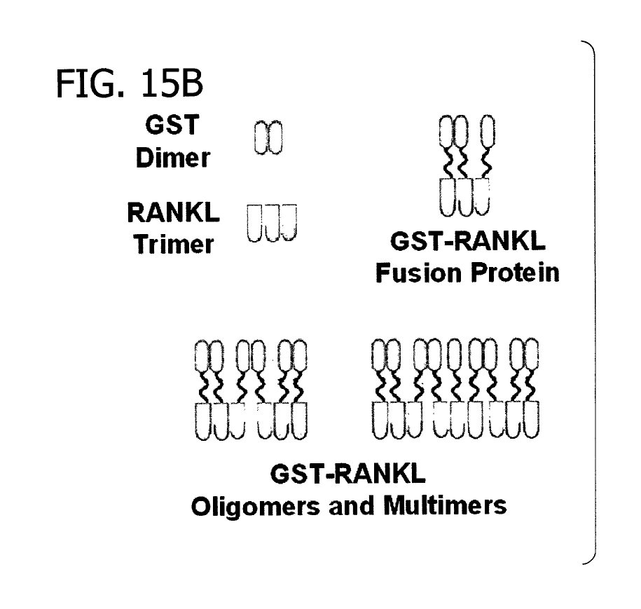

GST-RANKL was subjected to proteolysis to isolate the cleaved RANKL fragment from its GST fusion partner. Briefly, GST-RANKL was incubated with the type-14 human rhinovirus 3C protease (Amersham Pharmacia Biotech) for 4 hours at 4° C. in 50 mM Tris-HCl, pH 7.0,150 mM NaCl, 10 mM EDTA, and 1 mM DTT. Uncleaved fusion protein and GST-tagged protease were removed by passage over a glutathione affinity matrix. All purified recombinant proteins were assayed for endotoxin contamination by limulus amoebocyte lysate assay (Bio Whittaker), and analyzed by mass spectrometry to confirm identity. Both GST-RANKL and cleaved RANKL were dialyzed against physiologic salt and pH, and fractionated by gel filtration in Superose-626/60 using an AKTA explorer chromatography system (Amersham Pharmacia). Elution volumes were calibrated to molecular weight using the following standards: ribonuclease A (13,700), chymotrypsinogen A (25,000), ovalbumin (43,000), bovine serum albumin (67,000), aldolase (158,000), catalase (232,000), ferritin (440,000), thyroglobulin (669,000), and blue dextran 2000 (2,000,000). Fractions containing protein from different elution volumes were subjected to Western analysis using a monoclonal anti-GST primary antibody. As FIG. 15(a) shows, cleaved RANKL migrated as a single trimeric species (1 n), whereas GST-RANKL migrated as a polydisperse mixture of non-covalently associated mono-trimeric (1 n) and oligomeric (2-100 n) under dynamic equilibrium. Crystallographic evidence has established that GST possesses an innate tendency to dimerize, while RANKL spontaneously trimerizes. A single GST-RANKL trier, consisting of 3 RANKL molecules and 3 GST molecules, thus contains a free GST that is not bound to a neighboring GST, resulting in a 3:2 stoichiometry that engenders a propensity to oligomerize. High-order, branched oligomers form when the GST of a given GST-RANKL trimer forms a dimer with the GST from a neighboring GST-RANKL trimer (see FIG. 15(b)).

### Example 13

Internalization of GST-RANKL.

Primary murine osteoblasts were maintained in α-MEM containing 10% fetal bovine serum, and cultured in MEM containing 15% FBS, 50 μM ascorbic acid, and 10 mM β-glycerophosphate for differentiation. Cells were maintained at 37° C. in a humidified atmosphere containing 6% CO₂, with daily replenishment of media and cytokines. Primary murine osteoblasts were cultured on coverslips in A-MEM containing 10% fetal bovine serum and treated with GST-RANKL or cleaved RANKL for the indicated times. For phospholipid membrane staining, cells were incubated for 20 minutes with Vybrant Dil lipophilic carbocyanine membrane fluorescent stain (Molecular Probes). Cells were fixed in 4% paraformaldehyde, permeabilized with 0.1% Triton-X, blocked with 1% BSA/0.2% nonfat dry milk in PBS, and stained for RANK with a polyclonal anti-RANK antibody. Serial optical sections were obtained using a Radiance2100 laser scanning confocal microscope (BioRad). Microscope settings were calibrated to black level values using cells stained with an isotypic Ig control. GST-RANKL was cleaved as described in Example 12.

Primary osteoblasts in culture were exposed to 5 nM cleaved RANKL or GST-RANKL. At the indicated times, the cell surface was stained with a lipophilic fluorescent dye, and RANK was stained with an anti-RANK antibody. Confocal microscopy was employed to localize RANK (green fluorescence) and the cell surface (red fluorescence). On the merged images, colocalization of RANK and the cell surface appears yellow (overlap of green and red fluorescence). GST-RANKL:RANK complexes remain on the cell surface for at least one hour, corresponding to the sustained intracellular RANK signaling. In contrast, cleaved RANKL-RANK complexes are completely internalized within one hour, correlating to the absence of cleaved RANKL-induced RANK signaling at that time. Results are shown in FIG. 16.

### Example 14

Expression of Type I Collagen and Cbfa1 in Response to GST-RANKL.

For in vivo experiments, mice were administered 5 μg/kg GST-RANKL or GST alone as a control by subcutaneous injection and euthanized one hour later. For in vitro experimentation, primary osteoblasts were exposed to 100 ng/ml GST-RANKL or GST alone as a control. RNA was isolated with the RNeasy Total RNA System (Qiagen) and digested with deoxyribonuclease to eliminate genomic DNA. Meesenger RNA was subsequently isolated from total RNA with the Oligotex mRNA Purification System (Qiagen) and analyzed with the Platinum Quantitative RT-PCR Thermoscript One-Step System (Life Technologies). Briefly, 1 μg mRNA was reverse-transcribed to cDNA using murine gene-specific oligonucleotide primers designed to span exon-intron boundaries: Cbfa1 sense 5′-CCGCACGACMCCGCACCAT-3′ (SEQ ID NO. 3), Cbfa1 antisense 5′-CGCTCCGGCCCACAAATCTC-3′ (SEQ ID NO. 4), and Collagen type I chain α₁ sense 5′-TCTCCACTCTTCTAGTTCCT-3′ (SEQ ID NO. 5) and Colagen type I chain α₁ antisense 5′-TTGGGTCATTTCCACATGC-3′ (SEQ ID NO. 6). Reverse transcription was performed at 60° C. for 30 minutes, followed by denaturation at 95° C. for 5 minutes. Touchdown PCR amplification immediately ensued. As control, expression levels of hypoxanthine phosphoribosyl transferase (HPRT) were assessed concomitantly. Reaction products were fractionated electrophoretically in 2% agarose, and results were presented from the linear range of the assay.

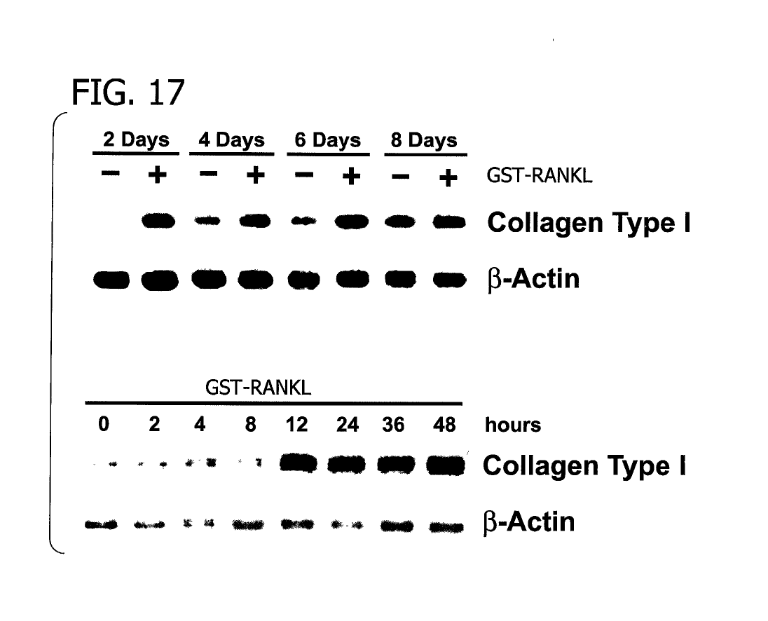

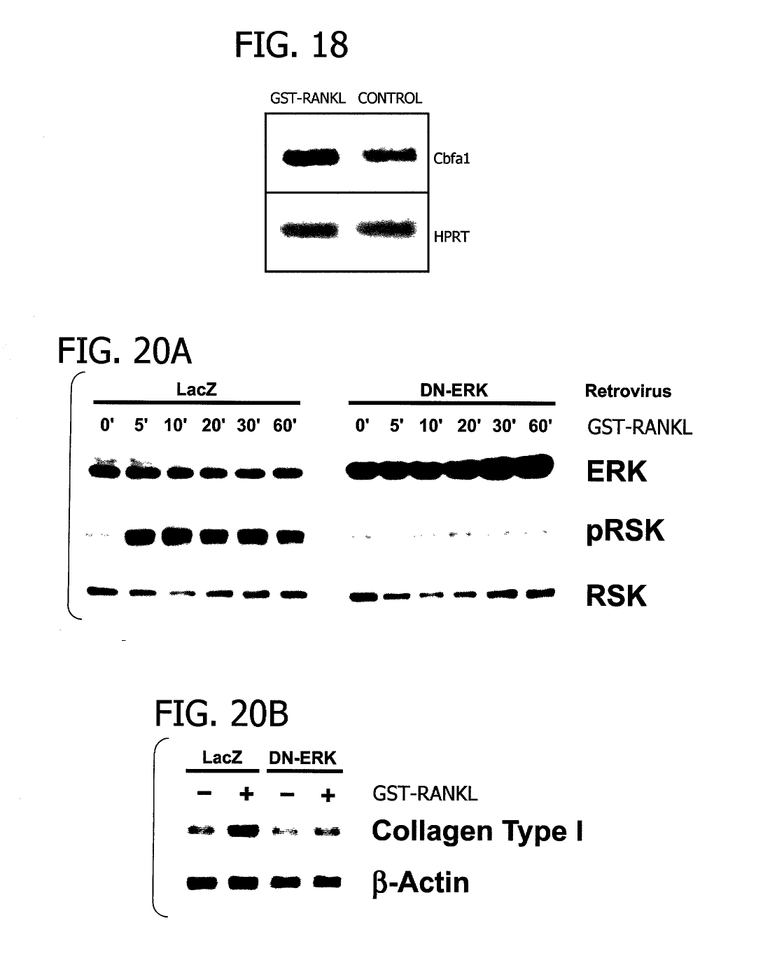

Type I collagen, synthesized by osteoblasts, is the major organic component of bone. As shown in FIG. 17, primary osteoblasts gradually upregulate collagen expression as they differentiate in culture. Intermittent GST-RANKL exposure accelerates this process, inducing robust collagen expression within 12 hours of initial exposure to it. Cbfa1 is the master transcription factor for osteoblastogenesis, and its absence results in a complete lack of osteoblasts and bone formation in mice (see, e.g., Otto et al., Cell 89, pp.765-771, 1997, and Komori et al., Cell 89, pp. 755-764, 1997). As shown in FIG. 18, expression of Cbfa1 is enhanced in the marrow within one hour of systemic GST-RANKL administration relative to the expression of control animals receiving GST alone.

### Example 15

GST-RANKL Stimulates Osteoblast Proliferation.

The proliferation rate of osteoblasts in vitro was assessed by incorporation of 5-bromo-2′-deoxyuridine (brdU) into DNA. Briefly, cells were cultured in the presence of 10 μM BrdU for 48 hours, in the presence or absence of 100 ng/ml GST-RANKL, or a molar equivalent of GST alone as control. BrdU incorporation was quantitated by ELISA (Amersham Pharmacia Biotech) using a peroxidase-labelled anti-BrdU antibody. Spectrophotometric measurement was performed at 450 nm following addition of the colorimetric substrate 3,3′-5,5′-tetramethylbenzidine.

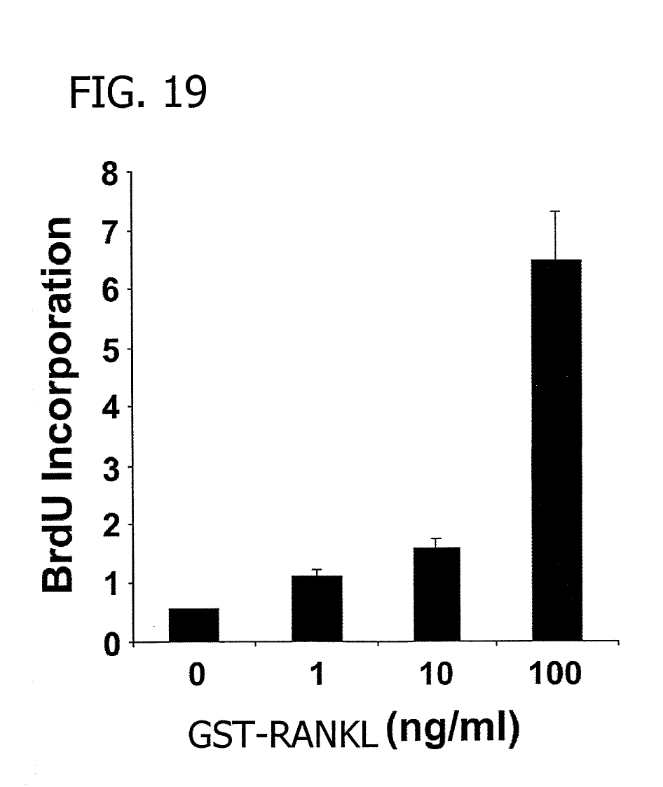

As shown in FIG. 19, GST-RANKL treatment enhanced the rate of osteoblast proliferation by up to 4-fold during a 48-hour assay period.

### Example 16

ERK Activation is Involved in Anabolic Effects of GST-RANKL.

A kinase-defective ERK1 cDNA (see Robbins et al., J. Biol. Chem., 268, pp.5097-5106, 1993) used in this experiment was a result of mutating alanine nucleotides at positions 211 and 212 to cytosine and guanine, respectively, resulting in replacement of tysine 71 with arginine (Erk1 K71R). ERK1 K71 R functions in a dominant-negative fashion to block both ERK1 and ERK2 activities (see Li et al., Immunol., 96, pp.524-528, 1999). The ERK1 K71R cDNA was cloned into the NcoI and BamHI restriction endonuclease sites of the SFG retroviral vector as described previously (see Ory et al., Proc. Natl. Acad. Sci. USA, 93, pp. 11400-11406, 1996). For generation of retroviral particles pseudotyped with vesicular stomatitis virus (VSV)-G glycoprotein, the SFG-ERK1 K71 R retroviral vector was transfected into a 293GPG packaginig cell line that expresses Mul V gag-pol and VSV-G glycoprotein under tetracycline regulation. Conditioned medium was harvested following tetracycline withdrawal from days 3 to 7, and found to contain a viral titer ≧5×10⁶ colony forming units/ml. Before transduction, the medium was filtered through a 0.45 μm membrane, and hexadimethrine bromide (polybrene) was added to a concentration of 8 μg/ml. As a negative control, a retrovirus carrying a LacZ cDNA was generated in the same fashion. Transduction with VSV-pseudotyped retroviri has been shown to exert no imact on osteobalst differentiation or function (see Kalajzic et al., Virology, 284, pp.37-45, 2001 and Liu et al., Bone 29, pp.331-335, 2001). For retroviral transduction, primary murine osteoblasts were cultured at a density of 60 cells per mm² in 150-mm culture dishes, and exposure to 25 ml of conditioned medium containing ≧5×10⁶ colony forming units/ml was allowed for 24 hours. Transduction efficiency exceeded 90%, as evidenced by X-gal staining of osteoblasts transduced with the LacZ retrovirus.

As seen in FIG. 20(a), osteoblasts transduced with dominant-negative ERK failed to phosphorylate RSK, a known downstream ERK substrate in response to a treatment with GST-RANKL. In addition, FIG. 20(b) shows that osteoblasts transduced with dominant-negative ERK failed to upregulate expression of type I collagen in response to GST-RANKL.

### Example 17

Expression of RANKL as an AP-RANKL Fusion Protein.

cDNA encoding murine RANKL residues 158-316 is cloned into the appropriate vector using the appropriate restriction endonucleases. A cDNA encoding the human alkaline phosphatase 1 is isolated from a cDNA library and spliced upstream (at amino terminal) of a RANKL cDNA in a suitable mammalian expression vector, such as, e.g., pcDNA3.1, using appropriate restriction endonucleases, such that the resulting DNA sequence is in frame, with no intervening stop codons. The resulting vector is transduced into a mammalian cell line, suce as, e.g., CHO cells by standard methods. Purified AP-RANKL is then assayed for endotoxin contamination by limulus amoebocyte lysate assay, and quantitated for bioactivity by an in vitro osteoclastogenesis readout. Human AP 1 is a secreted protein, and as a result, AP fusion protein is secreted into the media. After the sufficient amount of time for the AP-RANKL to be expressed and secreted by mammalian cells in vitro, the media is affinity purified to isolate AP-RANKL. The empirical mass of the AP-RANKL fusion protein is determined by mass spectrometry. The ability of AP-RANKL to form oligomeric complexes is checked by size exclusion chromatography.

### Example 18

Expression of RANKL as a GCN4-RANKL Fusion Protein.

cDNA encoding murine RANKL residues 158-316 is cloned into the appropriate vector using the appropriate restriction endonucleases. A DNA sequence encoding the GCN4 peptide is spliced upstream (at amino terminal) of a RANKL cDNA in a suitable expression vector, such as, e.g., pGEX-6P-1 (Accession No. U78872), using appropriate restriction endonucleases, such that the resulting DNA sequence is in frame, with no intervening stop codons. Following IPTG-mediated (0.05 mM) induction of protein expression in BL21 (DE3) Escherischia coli (Invitrogen), cells are triturated into a lysis buffer comprising 150 mM NaCl, 20 mM Tris-HCl pH 8.0, and 1 mM EDTA. Lysates are affinity purified to isolate GCN4—RANKL fusion protein. The isolated protein is then subjected to ion exchange chromatography, eluted with a salt gradient ranging from 0-500 mM NaCl, and dialyzed against physiologic salt and pH. Purified GCN4-RANKL is then assayed for endotoxin contamination by limulus amoebocyte lysate assay, and quantitated for bioactivity by an in vitro osteoclastogenesis readout.

The empirical mass of the GCN4-RANKL fusion protein is determined by mass spectrometry. The ability of GCN4-RANKL to form oligomeric complexes is checked by size exclusion chromatography.

### Example 19

Expression of a RANKL Derivative Comprising the TALL-1 Flap Region.

Murine RANKL containing residues 158-316 is mutated so that its DE loop (amino acids 245-249 containing the amino acid sequence SIKIP) is substituted with the DE loop of TALL-1 (amino acid sequence KVHVFGDEL). The mutations can be introduced into RANKL by PCR-driven site-directed mutagenesis, using the QuickChange Multi-Site Directed Mutagenesis Kit (available from Stratagene). The mutated RANKL is cloned into the appropriate vector, such as, e.g., pGEX-6P-1 (Accession No. U78872) using the appropriate restriction endonucleases such that the resulting DNA sequence is in frame, with no intervening stop codons. Following IPTG-mediated (0.05 mM) induction of protein expression in BL21 (DE3) Escherischia coli (Invitrogen), cells are triturated into a lysis buffer comprising 150 mM NaCl, 20 mM Tris-HCl pH 8.0, and 1 mM EDTA. Lysates are incubated with glutathione sepharose (Amersham) for affinity purification of the mutated RANKL protein, followed by excessive washing with buffer comprising 150 mM NaCl and 20 mM Tris-HCl pH 8.0. Following competitive elution (10 mM reduced glutathione) from the affinity column. The isolated protein is then subjected to ion exchange chromatography, eluted with a salt gradient ranging from 0-500 mM NaCl, and dialyzed against physiologic salt and pH. Purified RANKL derivative is then assayed for endotoxin contamination by limulus amoebocyte lysate assay, and quantitated for bioactivity by an in vitro osteociastogenesis readout.

The empirical mass of the mutant RANKL is determined by mass spectrometry. The ability of mutated RANKL to form oligomeric complexes is checked by size exclusion chromatography.

### Example 20

Expression of a RANKL Derivative Comprising the TALL-1 Flap Region and Additional Amino Acid Changes.

Murine RANKL containing residues 158-316 is mutated so that its DE loop (amino acids 245-249 containing the amino acid sequence SIKIP) is substituted with the DE loop of TALL-1 (amino acid sequence KVHVFGDEL). The following amino acid changes are made throughout the RANKL molecule to increase the similarity with the TALL-1 structure: 168T→I, 187Y→L, 194K→F, 212F→Y, 252H→V, 279F→I, and 283R→E. The mutations can be introduced into RANKL by PCR-driven site-directed mutagenesis, using the QuickChange Multi-Site Directed Mutagenesis Kit (available from Stratagene). The mutated RANKL is cloned into the appropriate vector, such as, e.g., pGEX-6P-1 using the appropriate restriction endonucleases such that the resulting DNA sequence is in frame, with no intervening stop codons. Following IPTG-mediated (0.05 mM) induction of protein expression in BL21 (DE3) Escherischia coli (Invitrogen), cells are triturated into a lysis buffer comprising 150 mM NaCl, 20 mM Tris-HCl pH 8.0, and 1 mM EDTA. Lysates are incubated with glutathione sepharose (Amersham) for affinity purification of the mutated RANKL protein, followed by excessive washing with buffer comprising 150 mM NaCl and 20 mM Tris-HCl pH 8.0. Following competitive elution (10 mM reduced glutathione) from the affinity column, The isolated protein is then subjected to ion exchange chromatography, eluted with a salt gradient ranging from 0-500 mM NaCl, and dialyzed against physiologic salt and pH. Purified RANKL derivative is then assayed for endotoxin contamination by limulus amoebocyte lysate assay, and quantitated for bioactivity by an in vitro osteoclastogenesis readout. The empirical mass of the mutant RANKL is determined by mass spectrometry. The ability of mutated RANKL to form oligomeric complexes is checked by size exclusion chromatography.

### Example 21

Ex vivo Stimulation of Bone Formation in Whole Calvarial Organ Culture.

An assay for bone formation is carried out as described in U.S. Pat. No. 6,080,779 col. 10, II. 29-55 incorporated herein by reference. Neo-natal mouse calvariae are placed in organ culture in the presence of vehicle, AP (a negative control), or increasing concentrations of purified AP-RANKL. Bone morphogenetic protein (BMP)-2 is administered as a positive control. Test compositions are administered for a period of 12 hours only at the initiation of the culture (1×) or once at initiation and once three days later, again for a duration of 12 hours (2×). After seven days, calvarial thickness is determined histomorphometrically and compared among the various control and experimental groups to assess bone formation.

### Example 22

In vivo Stimulation of Bone Formation in Mice.

Mice, C3H/HeN (Harlan, Indianapolis, Ind.) are administered 100 micrograms AP (control) or 100 micrograms AP-RANKL subcutaneously, once a day for nine days. Histological examination of tibia is then performed to assess the increase in bone mass and a net increase in the numbers of activated osteoblasts in AP-RANKL-treated as compared to control mice.

Dual-energy X-ray absorptiometry (DEXA) analysis of AP or AP-RANKL administered mice is also conducted using standard procedures to assess the change in bone mineral density in AP-RANKL mice compared to AP-treated mice.

### Example 23

Ex vivo Stimulation of Bone Formation in Whole Calvarial Organ Culture.

An assay for bone formation is carried out as described in U.S. Pat. No. 6,080,779 col. 10, II. 29-55 incorporated herein by reference. Neo-natal mouse calvariae are placed in organ culture in the presence of vehicle, GCN4 (a negative control), or increasing concentrations of purified GCN4-RANKL. Bone morphogenetic protein (BMP)-2 is administered as a positive control. Test compositions are administered for a period of 12 hours only at the initiation of the culture (1×) or once at initiation and once three days later, again for a duration of 12 hours (2×). After seven days, calvarial thickness is determined histomorphometrically and compared among the various control and experimental groups to assess bone formation.

### Example 24

In vivo Stimulation of Bone Formation in Mice.

Mice, C3H/HeN (Harlan, Indianapolis, Ind.) are administered 100 micrograms GCN4 (control) or 100 micrograms GCN4-RANKL subcutaneously, once a day for nine days. Histological examination of tibia is then performed to assess the increase in bone mass and a net increase in the numbers of activated osteoblasts in GCN4-RANKL-treated as compared to control mice.

Dual-energy X-ray absorptiometry (DEXA) analysis of GCN4 or GCN4-RANKL administered mice is also conducted using standard procedures to assess the change in bone mineral density in GCN4-RANKL mice compared to GCN4-treated mice.

### Example 25

Expression of RANKL as a GST-RANKL Fusion Protein.

cDNA encoding murine RANKL residues 158-316 was cloned into pGEX-6p-1 (Amersham; GenBank Accession No. U78872—see National Library of Medicine listing at http://ncbi.nlm.nih.gov under nucleic acids.) downstream of glutathione S-transferase using the SalI and NotI restriction endonucleases. Following IPTG-mediated (0.05 mM) induction of protein expression in BL21 (DE3) Escherischia coli (Invitrogen), cells were tritu rated into a lysis buffer comprising 150 mM NaCl, 20 mM Tris-HCl pH 8.0, and 1 mM EDTA. Lysates were incubated with glutathione sepharose (Amersham) for affinity purification of the GST-RANKL fusion protein, followed by excessive washing with buffer comprising 150 mM NaCl and 20 mM Tris-HCl pH 8.0. Following competitive elution (10 mM reduced glutathione) from the affinity column, the isolated protein was then subjected to ion exchange chromatography, eluted with a salt gradient ranging from 0-500 mM NaCl, and dialyzed against physiologic salt and pH. Purified GST-RANKL was then assayed for endotoxin contamination by limulus amoebocyte lysate assay, and quantitated for bioactivity by an in vitro osteoclastogenesis readout.

Under conditions replicating the physiological milieu, GST-RANKL formed i large oligomeric complexes, as demonstrated by size exclusion chromatography (data not shown). The majority of the protein existed as oligomeric complexes of GST-RANKL (data not shown).

### Example 26

Twenty, six week old C57BL/6 mice were randomly assigned to two experimental groups. Group 1 mice (10) received 100 ug injection of GST-RANKL in the intramedullary cavity of the right femur. Group 2 mice (10) received an equimolar volume injection of GST vehicle in the intramedullary cavity of the right femur.

Mice were anesthetized with a Ketamine/Xylazine cocktail (100 mg/kg ketamine and 10 mg/kg xylazine IP) and placed in left lateral recumbancy. The major trochanter and lateral femoral condyle of the right femur were identified and the intramedullary injection site was equidistant between these landmarks. The injections were made with 29 gauge needles on tuberculin syringes. On day 9, the mice were re-anesthetized with Ketamine/Xylazine cocktail (100 mg/kg ketamine and 10 mg/kg xylazine IP) and dual energy x-ray absorptiometry (DEXA, Piximus) analysis was done on each animal. Plain radiographs were taken immediately following DEXA analysis (Faxitron, KV 0.15, time=20 sec). Animals were sacrificed by CO₂ asphyxiation and both femurs harvested for histological analysis. The femurs were fixed in 10% buffered formalin for 48 hours and decalcified for 1 week. The DEXA analysis showed a significant difference in total bone mineral density (TBMD) between GST-RANKL-treated group and the control group (see Table 1). No significant difference was seen in either GST-RANKL or control group when comparing bone mineral density of the right and left femurs (see Table 2). There was no significant difference in skeletal density when comparing plain radiographs of both groups.

Table 1. BMD by Group

Means and standard deviations are reported. P-values test for significant differences between groups. They are based on unpaired t-tests.

TABLE 2
Femoral BMD by Side

| | Control | RANKL | p- |
|---|---|---|---|
| Variable | (n = 10) | (n = 10 | value |
| Total BMD (g/cm²) | 0.0529 ± 0.006 | 0.0656 ± 0.010 | 0.008 |
| Right femur BMD | 0.0543 ± 0.006 | 0.0632 ± 0.004 | 0.02 |
| (g/cm²) | | | |
| Left femur BMD | 0.0561 ± 0.007 | 0.0658 ± 0.007 | 0.03 |
| (g/cm²) | | | |

Means and standard deviations are reported for right and left femurs for each group. P-values test for significant differences between right and left sides. They are based on paired t-tests.

| | Right | Left | | |
|---|---|---|---|---|
| | Femur | Femur | Difference | |
| | BMD | BMD | (Right- | p- |
| Group | (g/cm²) | (g/cm²) | Left) | value |
| Control | 0.0543 ± | 0.0561 ± | −0.0018 ± | 0.06 |
| | 0.006 | 0.007 | 0.003 | |
| GS | 0.0632 ± | 0.0658 ± | −0.0026 ± | 0.49 |
| T-RANKL | 0.004 | 0.007 | 0.006 | |

Other features, objects and advantages of the present invention will be apparent to those skilled in the art. The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the present invention.

What is claimed is:

1. A method of enhancing processes of bone formation comprising administering an effective amount of an oligomeric complex of one or more of RANKL, a RANKL fusion protein, analog, derivative or mimic when bone formation is desired.

2. The method of claim 1 wherein the enhancement is selected from the group consisting of increasing activated osteoblast number and increasing osteoblast proliferation.

3. The method of claim 1 wherein the processes are selected from enhancement of osteoblast precursor differentiation and enhancement of osteoblast precursor proliferation.

4. The method of claim 1 wherein the desired bone formation comprises bone formation at a bone fracture site.

5. The method of claim 1 wherein the desired bone formation comprises bone formation at the junction of a bone and an allograft, autograft, bone prosthesis, or at a vertebral body fusion.

6. The method of claim 1 wherein the analog, derivative or mimic comprises a recombinant RANKL protein or fragment thereof.

7. The method of claim 1 wherein the fusion protein comprises GST-RANKL.

8. The method of claim 1 wherein the fusion protein comprises AP-RANKL.

9. The method of claim 1 wherein the fusion protein comprises leucine zipper-RANKL.

10. The method claim 1, wherein the RANKL derivative comprises RANKL protein comprising the flap region of TALL-1.

11. A method of treating a disease or condition manifested at least in part by the loss of bone mass comprising administering to a patient a pharmaceutical composition comprising an oligomeric complex of one or more of RANKL, a RANKL fusion protein, analog, derivative or mimic in an amount effective to promote bone formation and thereby prevent, inhibit or counteract said loss of bone mass.

12. The method of claim 11 wherein the pharmaceutical composition is administered intermittently.

13. The method of claim 11 wherein the patient is a mammal.

14. The method of claim 13 wherein the patient is human.

15. The method of claim 11 wherein the fusion protein comprises GST-RANKL.

16. The method of claim 11 wherein the fusion protein comprises AP-RANKL.

17. The method of claim 11 wherein the fusion protein comprises leucine zipper-RANKL.

18. The method of claim 11, wherein the RANKL derivative comprises RANKL protein comprising the flap region of TALL-1.

19. The method of claim 11 further comprising concomitant administration to said patient of a bone resorption inhibiting agent.

20. The method of claim 19 wherein the bone resorption inhibiting agent is selected from the group consisting of a bisphosphonate, a calcitonin, a calcitriol, an estrogen, a SERM and a calcium.

21. The method of claim 11 further comprising concomitant administration to said patient of one or more additional bone formation agents.

22. The method of claim 21 wherein one or more additional bone formation agents is selected from the group consisting of parathyroid hormone or its derivative, a bone morphogenetic protein, osteogenin, or a statin.

23. The method of claim 11 wherein the disease or condition is selected from the group consisting of osteoporosis, juvenile osteoporosis, osteogenesis imperfecta, hypercalcemia, hyperparathyroidism, osteomalacia, osteohalisteresis, osteolytic bone disease, osteonecrosis, Paget's disease, rheumatoid arthritis, inflammatory arthritis, osteomyelitis, corticosteroid treatment, periodontal disease, skeletal metastasis, cancer, age-related bone loss, osteopenia, and degenerate joint disease.

24. A composition for stimulating bone formation comprising an effective amount of an oligomeric complex of one or more of RANKL, a RANKL fusion protein, analog, derivative or mimic.

25. The composition of claim 24, further comprising a pharmaceutically acceptable excipient or carrier.

26. The composition of claim 24 wherein the stimulation of bone formation is selected from the group consisting of increasing activated osteoblast number and increasing osteoblast proliferation.

27. The composition of claim 24 wherein the stimulation of bone formation is selected from enhancement of osteoblast precursor differentiation and enhancement of osteoblast precursor proliferation.

28. The composition of claim 24 wherein the stimulation of bone formation comprises stimulation of bone formation at a bone fracture site.

29. The composition of claim 24 wherein the stimulation of bone formation comprises stimulation of bone formation at the junction of a bone and an allograft, autograft, bone prosthesis, or at a vertebral body fusion.

30. The composition of claim 24 wherein the analog, derivative or mimic comprises a recombinant RANKL protein or fragment thereof.

31. The composition of claim 24 wherein the fusion protein comprises GST-RANKL.

32. The composition of claim 24 wherein the fusion protein comprises AP-RANKL.

33. The composition of claim 24 wherein the fusion protein comprises leucine zipper-RANKL.

34. The composition of claim 24 wherein the RANKL derivative comprises RANKL protein comprising the flap region of TALL-1.

35. The composition of claim 24 further comprising one or more bone resorption inhibiting agents.

36. The composition of claim 35 wherein the bone resorption inhibiting agent is selected from the group consisting of a bisphosphonate, a calcitonin, a calcitriol, an estrogen, a SERM and a calcium.

37. The composition of claim 24 further comprising one or more additional bone formation agents.

38. The composition of claim 37 wherein one or more additional bone formation agents is selected from the group consisting of parathyroid hormone or its derivative, a bone morphogenetic protein, osteogenin, or a statin.
